(19) United States
(12) Patent Application Publication
(10) Pub. No.: US 2026/0295016 A1
(43) Pub. Date: Oct. 1, 2026

(54) NEUROTRAUMA TREATMENT

(71) Applicant: ECOLE POLYTECHNIQUE FEDERALE DE LAUSANNE (EPFL), Lausanne (CH)

(72) Inventors: Gregoire COURTINE, Lausanne (CH); Mark ANDERSON, Geneve (CH); Jordan SQUAIR, Lausanne (CH); Bernard SCHNEIDER, Belmont-Lausanne (CH)

(21) Appl. No.: 19/480,884

(22) PCT Filed: May 8, 2024
(86) PCT No.: PCT/EP2024/062688

§ 371 Date: Nov. 3, 2025

(30) Foreign Application Priority Data
May 8, 2023 (EP) 23172002.0
Sep. 22, 2023 (EP) 23199045.8

(51) Int. Cl. A61K 38/30; A61K 38/18; A61K 38/19; A61K 38/22; A61K 48/00; A61P 25/28

(52) U.S. Cl. CPC A61K 38/30; A61K 38/1808; A61K 38/1825; A61K 38/185; A61K 38/1866; A61K 38/19; A61K 38/22; A61K 48/0033; A61K 48/005; A61P 25/28

(57) ABSTRACT

The invention provides pharmaceutical compositions, combinations and vectors, such as expression vectors, as well as use thereof in a method for central nervous system repair after neurotrauma, such as spinal cord injury (SCI) or stroke and in a method for regenerating spinal cord or brain neurons within a subject having a spinal cord injury (SCI), stroke or other neurotraumatic conditions.

### FIELD OF THE INVENTION

The invention provides pharmaceutical compositions, combinations and vectors, such as expression vectors, as well as use thereof in a method for central nervous system repair after neurotrauma, such as spinal cord injury (SCI) or stroke and in a method for regenerating spinal cord or brain neurons within a subject having a spinal cord injury (SCI), stroke or other neurotraumatic conditions.

### BACKGROUND OF THE INVENTION

Spinal cord injury occurs when axons or nerve fibers of the spinal cord are interrupted, generally by mechanical forces. If the spinal cord is compressed, severed or contused, the axons may be physically or physiologically disintegrated, so that no conduction of neuroelectric impulses can occur along the affected axon's length. Eventually, large populations of axons and their associated cell bodies may die, causing massive loss in communication between the brain and the peripheral nerves, resulting in varying degrees of functional deficit. Currently available therapies have demonstrated only limited success in restoring function following spinal cord injury.

A similar neuropathological response occurs following stroke, in which a broken or ruptured blood vessel in the brain breaks, resulting in often times large non-neural fibrotic lesion cores, accompanied by disruption of axonal connectivity and permanent loss of neurological function.

A consequence of spinal cord injury is the formation of scar tissue and posttraumatic microcystic myelomalacia. The extent of each of these post-injury maladies depends on injury severity. Two types of scar tissue can be formed, namely, glial and fibrous. The glial scar consists of a loose network of astrocytic processes connected by tight junctions. There has recently been a challenge to a prevailing dogma that glial scars are regarded as a failure of axonal regrowth in the central nervous system. Instead, astrocytes in spinal cord injury lesions were found to express multiple axon-growth-supporting molecules responsible for stimulating axonal regrowth past scar-forming astrocytes. Fibrous scarring is made up of extracellular matrix deposition and type IV collagen, which form a tight barrier. In addition to creating a mechanical barrier, both types of scars may obstruct neuronal regeneration. Current tissue engineering research is focused on constructing a permissive environment at the site of injury that would support axonal regeneration.

To date, no treatment option exists that is able to promote healing, reduce fibrous scarring, ameliorate cellular damage following spinal cord injury or stroke, provide neuroprotection or induce the growth and development of spinal cord cells to replace damaged or dead cells, any or all of which could help return the injured patient to normal or near normal function. Therefore, it is an object of the instant invention to provide such treatment options for spinal cord injury or stroke patients.

### SUMMARY OF THE INVENTION

An aspect of the present invention provides a pharmaceutical combination comprising
i) a first agent comprising exogenous nucleic acid encoding an osteopontin (Spp1) polypeptide as set forth in SEQ ID NO: 1 or SEQ ID NO: 8, insulin-like growth factor 1 (Igf1) polypeptide as set forth in SEQ ID NO: 2 and ciliary-derived neurotrophic factor (Cntf) polypeptide as set forth in SEQ ID NO: 3 or SEQ ID NO: 9; and/or
ii) a second agent comprising exogenous nucleic acid encoding a fibroblast growth factor 2 (FGF2) polypeptide as set forth in SEQ ID NO: 4 or SEQ ID NO: 10, epidermal growth factor (EGF) polypeptide as set forth in SEQ ID NO: 5 or SEQ ID NO: 11, and a glial-derived neurotrophic factor (GDNF) polypeptide as set forth in SEQ ID NO: 6;
wherein, when both present, said first and second agents are administered sequentially or simultaneously, and
wherein said first and second agents provide sustained expression and secretion of effective amounts of said polypeptides during at least 3 days.

Another aspect of the present invention provides a pharmaceutical combination of the invention for use in a method of treating an incomplete spinal cord injury or a stroke in a subject.

Another aspect of the present invention provides a pharmaceutical combination of the invention for use in a method of treating a complete spinal cord injury in a subject, wherein the pharmaceutical combination comprises the first agent, the second agent and the third agent.

Further embodiments of the present invention are defined by the appended claims.

The above and other objects, features and advantages of the herein presented subject-matter will become more apparent from a study of the following description with reference to the attached figures showing some preferred aspects of said subject-matter.

### BRIEF DESCRIPTION OF THE FIGURES

FIG. 1 shows identification of Vsx2 neurons. Clustering tree of neuronal subpopulations in the thoracic spinal cord. VEP Vsx2 neurons undergo the most profound transcriptional change following incomplete SCI.

FIG. 2 shows projection pattern of long projecting Vsx2 neurons. Image of the spinal cord virally traced to label long projecting Vsx2 neurons, which terminate in the lumbar spinal cord.

FIG. 3 shows walking not restored when guiding axons immediately past SCI lesions. (Top) Axons are guided one segment past SCI lesions using biomaterial depots of Gdnf and are unable to restore walking. (Bottom) Axons are guided two segments past SCI lesions using biomaterial depots of Gdnf, resulting in longer but less dense growth and also failing to restore walking.

FIG. 4 shows axon regeneration following complete SCI. Gene therapy based regenerative intervention regrows axons past SCI lesions and into the lumbar spinal cord.

FIG. 5 shows loss of function experiment of Vsx2 neurons. Viral ablation of Vsx2 neurons after regeneration diminishes walking ability.

FIG. 6 shows lentiviral delivery of growth factors after SCI. Delivering agents via singular lentiviral constructs is able to induce axon regeneration and restoration of walking when delivered after the SCI.

FIG. 7 shows axon growth in moderate SCI. Delivery of AAV-Igf1/Cntf/Spp1, on its own, induces growth of propriospinal axons following incomplete crush SCI.

FIG. 8 shows acceleration of astrocyte border in old mice following crush SCI. Lentiviral delivery of Egf/Fgf2/Vegf restricts lesion size and restores walking following SCI in old mice.

FIG. 9 shows acceleration of astrocyte border in old mice following ischemic stroke. Lentiviral delivery of Egf/Fgf2/Vegf restricts lesion size following ischemic stroke in old mice.

FIG. 10 shows lentiviral plasmid maps. Plasmid maps of lentivirus constructs using either GFAP or PGK promoters.

### DETAILED DESCRIPTION OF THE INVENTION

All, documents, patents, patent applications, publications, product descriptions, and protocols which are cited throughout this application are incorporated herein by reference in their entireties for all purposes. The publications and applications discussed herein are provided solely for their disclosure prior to the filing date of the present application. Nothing herein is to be construed as an admission that the present invention is not entitled to antedate such publication by virtue of prior invention. In addition, the materials, methods, and examples are illustrative only and are not intended to be limiting.

In the case of conflict, the present specification, including definitions, will control. Unless defined otherwise, all technical and scientific terms used herein have the same meaning as is commonly understood by one of skill in the art to which the subject matter herein belongs. As used herein, the following definitions are supplied in order to facilitate the understanding of the present invention.

The term “comprise” is generally used in the sense of include, that is to say permitting the presence of one or more features or components. Also as used in the specification and claims, the language “comprising” can include analogous embodiments described in terms of “consisting of” and/or “consisting essentially of”.

As used in the specification and claims, the singular form “a”, “an” and “the” include plural references unless the context clearly dictates otherwise.

As used in the specification and claims, the term “and/or” used in a phrase such as “A and/or B” herein is intended to include “A and B”, “A or B”, “A”, and “B”.

As used herein, the terms “treat”, “treatment”, “treating” or “amelioration” refer to therapeutic treatments, wherein the object is to reverse, alleviate, ameliorate, inhibit, slow down or stop the progression or severity of a condition associated with a spinal cord injury or stroke. The term “treating” includes reducing or alleviating at least one adverse effect or symptom of a spinal cord injury, e.g., partial or complete paralysis. Treatment is generally “effective” if one or more symptoms or clinical markers are reduced. Alternatively, treatment is “effective” if the progression of a disease is reduced or halted. That is, “treatment” includes not just the improvement of symptoms or markers, but also a cessation of, or at least slowing of, progress or worsening of symptoms compared to what would be expected in the absence of treatment. Beneficial or desired clinical results include, but are not limited to, alleviation of one or more symptom(s), diminishment of extent of disease, stabilized (i.e., not worsening) state of a spinal cord injury or stroke, delay or slowing of a spinal cord injury progression, amelioration or palliation of the injury state, remission (whether partial or total), and/or decreased mortality, whether detectable or undetectable. The term “treatment” of a spinal cord injury or stroke also includes providing relief from the symptoms or side-effects of the disease (including palliative treatment).

As used herein, the term “administering” refers to the placement of a therapeutic (e.g., an agent, a viral vector etc.) or pharmaceutical combination or composition as disclosed herein into a subject by a method or route which results in at least partial delivery of the agent to the subject. Pharmaceutical combinations or compositions comprising agents as disclosed herein can be administered by any appropriate route which results in an effective treatment in the subject.

As used herein, a “subject” means a human or animal. Usually the animal is a vertebrate such as a primate, rodent, domestic animal or game animal. Primates include, for example, chimpanzees, cynomologous monkeys, spider monkeys, and macaques, e.g., Rhesus. Rodents include, for example, mice, rats, woodchucks, ferrets, rabbits and hamsters. Domestic and game animals include, for example, cows, horses, pigs, deer, bison, buffalo, feline species, e.g., domestic cat, canine species, e.g., dog, fox, wolf, avian species, e.g., chicken, emu, ostrich, and fish, e.g., trout, catfish and salmon. In some embodiments, the subject is a mammal, e.g., a primate, e.g., a human. The terms, “individual”, “patient” and “subject” are used interchangeably herein. Preferably, the subject is a mammal. The mammal can be a human, non-human primate, mouse, rat, dog, cat, horse, or cow, but is not limited to these examples. Mammals other than humans can be advantageously used as subjects that represent animal models of spinal cord injury. A subject can be male or female. A subject can be one who has been previously diagnosed with or identified as suffering from or having a spinal cord injury or one or more complications related to such an injury, and optionally, have already undergone treatment for a spinal cord injury or the one or more complications related to the injury. Alternatively, a subject can also be one who has not been previously diagnosed as having such spinal cord injury or related complications. For example, a subject can be one who exhibits one or more risk factors for a spinal cord injury or stroke, e.g., participates in an activity that is likely to result in a spinal cord injury or stroke, for example, a full contact sport, e.g., American football, or one or more complications related to spinal cord injury or stroke or a subject who does not exhibit risk factors. Subjects having or being suspected to have a spinal cord injury or stroke can be identified by a physician using current methods of diagnosing a condition.

Symptoms and/or complications of a spinal cord injury or stroke, which characterize this injury and aid in diagnosis are well known in the art and include but are not limited to, loss or reduce mobility in limbs. Tests that may aid in a diagnosis of a spinal cord injury, include but are not limited to an x-ray, an MRI scan, a myelogram, somatosensory evoked potential (SSEP) testing or a CT scan.

Methods, pharmaceutical combinations and compositions described herein are used for the treatment of a spinal cord injury or stroke. As used herein, a “spinal cord injury” or “SCI” refers to any insult to any region of the spinal cord, e.g., the cervical vertebrae, the thoracic vertebrae, the lumbar vertebrae, the sacral vertebrae, the sacrum, or the coccyx. A “spinal cord injury” can result in various levels of severity, ranging from no effect on mobility, e.g., retain walking ability, to paraplegia (e.g., paralysis of legs and lower region of body), and tretraplegia (e.g., loss of muscle strength in all four extremities). A “spinal cord injury” can be a complete spinal cord injury, e.g., an injury that produces total loss of all motor and sensory function below the site of injury. A “spinal cord injury” can be an incomplete spinal cord injury, e.g., in which some motor function remains below the primary site of the injury. Non-limiting examples of incomplete spinal cord injuries include, but are not limited to, anterior cord syndrome, centre cord syndrome, and Brown-Sequard syndrome. A “spinal cord injury” can be a spinal concussion or spinal contusion, e.g., an injury that resolves itself in, e.g., one or two days. A spinal concussion or contusion can be complete or incomplete.

Complete or incomplete spinal cord injury can be assessed and categorized based on the American Spinal Injury Association (ASIA) impairment scale or AIS, which describes a person's functional impairment as a result of a SCI. This scale indicates how much sensation a person feels after light touch and a pin prick at multiple points on the body and tests key motions on both sides of the body.

LT=light touch; PP=pin prick; DAP=deep anal pressure; AIS=ASIA Impairment Scale; NLI=neurological level of injury
Grade A=Complete. No sensory or motor function is preserved in the sacral segments S4-5.
Grade B=Sensory Incomplete. Sensory but no motor function is preserved below the neurological level and includes the sacral segments S4-5 (LT or PP at S4-5 or DAP), and no motor function is preserved more than three levels below the motor level on either side of the body.
Grade C=Motor Incomplete. Motor function is preserved at the most caudal sacral segments for voluntary anal contraction OR the patient meets the criteria for sensory incomplete status (sensory function preserved at the most caudal sacral segments (S4-S5) by LT, PP or DAP), and has some sparing of motor function more than three levels below the ipsilateral motor level on either side of the body. (This includes key or non-key muscle functions to determine motor incomplete status.) For AIS C—less than half of key muscle functions below the single NLI have a muscle grade ≥3 (i.e., they are not strong enough to move against gravity).
Grade D=Motor Incomplete. Motor incomplete status as defined above, with at least half (half or more) of key muscle functions below the single NLI having a muscle grade ≥3 (i.e., the joints can be moved against gravity).

Accordingly, in the frame of the present invention, when reference is made to “complete spinal cord injury” it is herein meant a spinal cord injury of Grade A according to the AIS, whereas for “incomplete spinal cord injury” it is herein meant a spinal cord injury of Grade B to D according to the AIS.

Spinal cord injury (SCI) severs the connections of axons from the brain to the spinal cord. Due to the limited ability of damaged axons to regrow across injuries, SCI typically leads to permanent neurological deficits. In certain types of incomplete SCI, rehabilitation can improve functional recovery by stimulating the reorganization of axons that have been spared by the incomplete injuries. In contrast, individuals that suffer from anatomically complete SCI have no spared axons that cross the injury site. Recovery from such complete injuries will require stimulating axons to regrow across the complete SCI lesion and to form new connections with neurons below the injury. One aim of the invention is to address this limitation in the art.

In order to address the above-mentioned limitation, a mechanism-based biological repair strategy is herein disclosed that is able to induce the regeneration of molecularly defined subpopulations of spinal cord neurons, guide them to reconnect with their natural topological targets, and restore overground walking in a subject with complete or incomplete spinal cord injury (SCI), or stroke. The inventors applied projection-specific and comparative single-nucleus RNA sequencing to uncover the transcriptional phenotype and connectome of neuronal subpopulations involved in natural spinal cord repair. The inventors identified a molecularly defined population of excitatory projection neurons in the thoracic spinal cord that extend axons to the lumbar spinal cord where walking execution centres reside. It is herein shown that regrowing axons from these specific neurons across anatomically complete SCI and guiding them to reconnect with their appropriate target region in the lumbar spinal cord restores walking in mice. These results demonstrate that mechanism-based repair strategies that recapitulate the natural topology of molecularly defined neuronal subpopulations can restore neurological functions. The inventors also demonstrate that failure of blood-brain-barrier and astrocyte border formation leads to exaggerated injury severity. Administration of growth factors that accelerate the formation of these barriers improves neurological and functional recovery.

Indeed, it is herein shown that phenocopying mechanisms underlying natural spinal cord repair can achieve recovery in mice with, for instance, anatomically complete SCI. Regrowing axons from a specific neuronal subpopulation across anatomically complete SCI and guiding them to reconnect with their appropriate target region in the lumbar spinal cord restores walking in mice with complete paralysis. As expected, treated mice did not walk as well as uninjured mice, but instead exhibited a comparable phenotype to mice that underwent natural spinal cord repair after incomplete SCI. Achieving comparable repair across the entire cellular architecture of the spinal cord will be facilitated by comprehensive molecular catalogues of all neuronal populations, their requirements for growth, and subpopulation-specific chemoattractants. Such catalogues may unlock the framework to achieve complete repair of the injured spinal cord and this principle may expedite repair after other forms of central nervous system injury and disease.

In view of the above, an aspect of the present invention provides a pharmaceutical combination comprising
i) a first agent comprising exogenous nucleic acid encoding an osteopontin (Spp1) polypeptide as set forth in SEQ ID NO: 1 or SEQ ID NO: 8, insulin-like growth factor 1 (Igf1) polypeptide as set forth in SEQ ID NO: 2 and ciliary-derived neurotrophic factor (Cntf) polypeptide as set forth in SEQ ID NO: 3 or SEQ ID NO: 9; and/or
ii) a second agent comprising exogenous nucleic acid encoding a fibroblast growth factor 2 (FGF2) polypeptide as set forth in SEQ ID NO: 4 or SEQ ID NO: 10, epidermal growth factor (EGF) polypeptide as set forth in SEQ ID NO: 5 or SEQ ID NO: 11, and a glial-derived neurotrophic factor (GDNF) polypeptide as set forth in SEQ ID NO: 6;
wherein, when both present, said first and second agents are administered sequentially or simultaneously, and
wherein said first and second agents provide sustained expression and secretion of effective amounts of said polypeptides during at least 3 days.

According to an embodiment of the pharmaceutical combinations of the invention, the second agent further comprises exogenous nucleic acid encoding a vascular endothelial growth factor (VEGF) polypeptide as set forth in SEQ ID NO: 7 or SEQ ID NO:12.

According to another embodiment, the pharmaceutical combinations of the invention further comprises
iii) a third agent comprising exogenous nucleic acid encoding a glial-derived neurotrophic factor (GDNF) polypeptide as set forth in SEQ ID NO: 6;
wherein said first, second and third agents are administered sequentially or simultaneously, and
wherein said first, second and third agents provide sustained expression and secretion of effective amounts of said polypeptides during at least 3 days.

According to an embodiment of the pharmaceutical combinations of the invention, the first agent, the second agent and the third agent is a gene editing system, a vector or expression vectors containing one or more nucleic acid sequences encoding the one or more polypeptides of the invention, described herein.

According to another embodiment of the pharmaceutical combinations of the invention, the expression vector is a viral vector selected from the group consisting of retrovirus, lentivirus, adenovirus, herpesvirus, poxvirus, alpha virus, vaccinia virus, and adeno-associated viruses.

According to another embodiment of the pharmaceutical combinations of the invention, a vector is an mRNA vector.

According to another embodiment of the pharmaceutical combinations of the invention, the first agent is a lentivirus expression vector, the second agent is an adeno-associated virus (AAV) expression vector and the third agent is an adeno-associated virus (AAV) expression vector, and wherein the adeno-associated virus (AAV) is selected from the group comprising AAV1, AAV2, AAV3, AAV5, AAV6, AAV7, AAV8, AAV9, AAV10 and AAV11.

According to another embodiment of the pharmaceutical combinations of the invention, the first and second agents are each a lentivirus expression vector. According to still another embodiment, the first, second and third agents are each a lentivirus expression vector.

According to another embodiment of the pharmaceutical combination of the invention, the third agent provides sustained glial-derived neurotrophic factor (GDNF) delivery.

According to another embodiment, the present invention provides a pharmaceutical combination comprising
i) a first agent consisting of
   a. a lentivirus vector comprising exogenous nucleic acid encoding an osteopontin (Spp1) polypeptide as set forth in SEQ ID NO: 1 or SEQ ID NO: 8,
   b. a lentivirus vector comprising exogenous nucleic acid encoding insulin-like growth factor 1 (Igf1) polypeptide as set forth in SEQ ID NO: 2, and
   c. a lentivirus vector comprising exogenous nucleic acid encoding ciliary-derived neurotrophic factor (Cntf) polypeptide as set forth in SEQ ID NO: 3 or SEQ ID NO: 9;
ii) a second agent consisting of
   a. a lentivirus vector comprising exogenous nucleic acid encoding a fibroblast growth factor 2 (FGF2) polypeptide as set forth in SEQ ID NO: 4 or SEQ ID NO: 10,
   b. a lentivirus vector comprising exogenous nucleic acid encoding epidermal growth factor (EGF) polypeptide as set forth in SEQ ID NO: 5 or SEQ ID NO: 11, and
   c. a lentivirus vector comprising exogenous nucleic acid encoding a glial-derived neurotrophic factor (GDNF) polypeptide as set forth in SEQ ID NO: 6; and
iii) a third agent consisting of a lentivirus vector comprising exogenous nucleic acid encoding a glial-derived neurotrophic factor (GDNF) polypeptide as set forth in SEQ ID NO: 6,
wherein, said first agent, said second agent and said third agent are administered sequentially or simultaneously, and wherein said first agent, said second agent and said third agent provide sustained expression and secretion of effective amounts of said polypeptides during at least 3 days.

Osteopontin (Spp1) polypeptide(sequence of over expressed Spp1 (mus
musculus):
(SEQ ID NO: 1)
MRLAVICFCLFGIASSLPVKVTDSGSSEEKLYSLHPDPIATWLVPDPSQKQNLLAPQN
AVSSEEKDDFKQETLPSNSNESHDHMDDDDDDDDDDGDHAESEDSVDSDESDESHH
SDESDETVTASTQADTFTPIVPTVDVPNGRGDSLAYGLRSKSRSFQVSDEQYPDATDE
DLTSHMKSGESKESLDVIPVAQLLSMPSDQDNNGKGSHESSQLDEPSLETHRLEHSKE
SQESADQSDVIDSQASSKASLEHQSHKFHSHKDKLVLDPKSKEDDRYLKFRISHELES
SSSEVNDYKDDDDK* (FLAG tag in italic letters)
Homo sapiens secreted phosphoprotein 1 (SPP1)
(SEQ ID NO: 8)
MRIAVICFCLLGITCAIPVKQADSGSSEEKQLYNKYPDAVATWLNPDPSQKQNLLAPQ
NAVSSEETNDFKQETLPSKSNESHDHMDDMDDEDDDDHVDSQDSIDSNDSDDVDDT
DDSHQSDESHHSDESDELVTDFPTDLPATEVFTPVVPTVDTYDGRGDSVVYGLRSKS
KKFRRPDIQYPDATDEDITSHMESEELNGAYKAIPVAQDLNAPSDWDSRGKDSYETS
QLDDQSAETHSHKQSRLYKRKANDESNEHSDVIDSQELSKVSREFHSHEFHSHEDML
VVDPKSKEEDKHLKFRISHELDSASSEVN*
Insulin-like growth factor 1 (Igf1) polypeptide(sequence of overexpressed
IGF1 (homo sapiens):
(SEQ ID NO: 2)
MGKISSLPTQLFKCCFCDFLKVKMHTMSSSHLFYLALCLLTFTSSATAGPETLCGAEL
VDALQFVCGDRGFYFNKPTGYGSSSRRAPQTGIVDECCFRSCDLRRLEMYCAPLKPA
KSARSVRAQRHTDMPKTQKEVHLKNASRGSAGNKNYRM*
Ciliary-derived neurotrophic factor (Cntf) polypeptide(sequence of
overexpressed Cntf (mus musculus):
(SEQ ID NO: 3)
MAFAEQSPLTLHRRDLCSRSIWLARKIRSDLTALMESYVKHQGLNKNISLDSVDGVP
VASTDRWSEMTEAERLQENLQAYRTFQGMLTKLLEDQRVHFTPTEGDFHQAIHTLTL
QVSAFAYQLEELMALLEQKVPEKEADGMPVTIGDGGLFEKKLWGLKVLQELSQWT
VRSIHDLR VISSHHMGISAHESHYGAKQMYPYDVPDYA* (HA tag in italic letters)
Homo sapiens ciliary neurotrophic factor (CNTF)
(SEQ ID NO: 9)
MAFTEHSPLTPHRRDLCSRSIWLARKIRSDLTALTESYVKHQGLNKNINLDSADGMP
VASTDQWSELTEAERLQENLQAYRTFHVLLARLLEDQQVHFTPTEGDFHQAIHTLLL
QVAAFAYQIEELMILLEYKIPRNEADGMPINVGDGGLFEKKLWGLKVLQELSQWTVR
SIHDLRFISSHQTGIPARGSHYIANNKKM
Fibroblast growth factor 2 (FGF2) polypeptide(sequence of overexpressed
Fgf2 (mus musculus):
(SEQ ID NO: 4)
MAASGITSLPALPEDGGAAFPPGHFKDPKRLYCKNGGFFLRIHPDGRVDGVREKSDP
HVKLQLQAEERGVVSIKGVCANRYLAMKEDGRLLASKCVTEECFFFERLESNNYNT
YRSRKYSSWYVALKRTGQYKLGSKTGPGQKAILFLPMSAKS*
Homo sapiens fibroblast growth factor 2 (FGF2)
(SEQ ID NO: 10)
MAAGSITTLPALPEDGGSGAFPPGHFKDPKRLYCKNGGFFLRIHPDGRVDGVREKSDP
HIKLQLQAEERGVVSIKGVCANRYLAMKEDGRLLASKCVTDECFFFERLESNNYNTY
RSRKYTSWYVALKRTGQYKLGSKTGPGQKAILFLPMSAKS*
Epidermal growth factor (EGF) polypeptide(sequence of overexpressed Egf
(mus musculus):
(SEQ ID NO: 5)
MAPKKKRKVRIMPWGRRPTWLLLAFLLVFLKISILSVTAWQTGNCQPGPLERSERSG
TCAGPAPFLVFSQGKSISRIDPDGTNHQQLVVDAGISADMDIHYKKERLYWVDVERQ
VLLRVFLNGTGLEKVCNVERKVSGLAIDWIDDEVLWVDQQNGVITVTDMTGKNSRV
LLSSLKHPSNIAVDPIERLMFWSSEVTGSLHRAHLKGVDVKTLLETGGISVLTLDVLD
KRLFWVQDSGEGSHAYIHSCDYEGGSVRLIRHQARHSLSSMAFFGDRIFYSVLKSKAI
WIANKHTGKDTVRINLHPSFVTPGKLMVVHPRAQPRTEDAAKDPDPELLKQRGRPCR
FGLCERDPKSHSSACAEGYTLSRDRKYCEDVNECATQNHGCTLGCENTPGSYHCTCP
TGFVLLPDGKQCHELVSCPGNVSKCSHGCVLTSDGPRCICPAGSVLGRDGKTCTGCS
SPDNGGCSQICLPLRPGSWECDCFPGYDLQSDRKSCAASGPQPLLLFANSQDIRHMHF
DGTDYKVLLSRQMGMVFALDYDPVESKIYFAQTALKWIERANMDGSQRERLITEGV
DTLEGLALDWIGRRIYWTDSGKSVVGGSDLSGKHHRIIIQERISRPRGIAVHPRARRLF
WTDVGMSPRIESASLQGSDRVLIASSNLLEPSGITIDYLTDTLYWCDTKRSVIEMANL
DGSKRRRLIQNDVGHPFSLAVFEDHLWVSDWAIPSVIRVNKRTGQNRVRLQGSMLKP
SSLVVVHPLAKPGADPCLYRNGGCEHICQESLGTARCLCREGFVKAWDGKMCLPQD
YPILSGENADLSKEVTSLSNSTQAEVPDDDGTESSTLVAEIMVSGMNYEDDCGPGGC
GSHARCVSDGETAECQCLKGFARDGNLCSDIDECVLARSDCPSTSSRCINTEGGYVCR
CSEGYEGDGISCFDIDECQRGAHNCAENAACTNTEGGYNCTCAGRPSSPGRSCPDST
APSLLGEDGHHLDRNSYPGCPSSYDGYCLNGGVCMHIESLDSYTCNCVIGYSGDRCQ
TLDLRWWELRHAGYGQKHDIMVVAVCMVALVLLLLLGMWGTYYYRTRKQLSNPP
KNPCDEPSGSVSSSGPDSSSGAAVASCPQPWFVVLEKHQDPKNGSLPADGTNGAVVD
AGLSPSLQLGSVHLTSWRQKPHIDGMGTGQSCWIPPSSDRGPQEIEGNSHLPSYRPVG
PEKLHSLQSANGSCHERAPDLPRQTEPVQ*
Homo sapiens epidermal growth factor (EGF)
(SEQ ID NO: 11)
MLLTLIILLPVVSKFSFVSLSAPQHWSCPEGTLAGNGNSTCVGPAPFLIFSHGNSIFRID
TEGTNYEQLVVDAGVSVIMDFHYNEKRIYWVDLERQLLQRVFLNGSRQERVCNIEK
NVSGMAINWINEEVIWSNQQEGIITVTDMKGNNSHILLSALKYPANVAVDPVERFIFW
SSEVAGSLYRADLDGVGVKALLETSEKITAVSLDVLDKRLFWIQYNREGSNSLICSCD
YDGGSVHISKHPTQHNLFAMSLFGDRIFYSTWKMKTIWIANKHTGKDMVRINLHSSF
VPLGELKVVHPLAQPKAEDDTWEPEQKLCKLRKGNCSSTVCGQDLQSHLCMCAEGY
ALSRDRKYCEDVNECAFWNHGCTLGCKNTPGSYYCTCPVGFVLLPDGKRCHQLVSC
PRNVSECSHDCVLTSEGPLCFCPEGSVLERDGKTCSGCSSPDNGGCSQLCVPLSPVSW
ECDCFPGYDLQLDEKSCAASGPQPFLLFANSQDIRHMHFDGTDYGTLLSQQMGMVY
ALDHDPVENKIYFAHTALKWIERANMDGSQRERLIEEGVDVPEGLAVDWIGRRFYW
TDRGKSLIGRSDLNGKRSKIITKENISQPRGIAVHPMAKRLFWTDTGINPRIESSSLQGL
GRLVIASSDLIWPSGITIDFLTDKLYWCDAKQSVIEMANLDGSKRRRLTQNDVGHPFA
VAVFEDYVWFSDWAMPSVMRVNKRTGKDRVRLQGSMLKPSSLVVVHPLAKPGADP
CLYQNGGCEHICKKRLGTAWCSCREGFMKASDGKTCLALDGHQLLAGGEVDLKNQ
VTPLDILSKTRVSEDNITESQHMLVAEIMVSDQDDCAPVGCSMYARCISEGEDATCQC
LKGFAGDGKLCSDIDECEMGVPVCPPASSKCINTEGGYVCRCSEGYQGDGIHCLDIDE
CQLGEHSCGENASCTNTEGGYTCMCAGRLSEPGLICPDSTPPPHLREDDHHYSVRNS
DSECPLSHDGYCLHDGVCMYIEALDKYACNCVVGYIGERCQYRDLKWWELRHAGH
GQQQKVIVVAVCVVVLVMLLLLSLWGAHYYRTQKLLSKNPKNPYEESSRDVRSRRP
ADTEDGMSSCPQPWFVVIKEHQDLKNGGQPVAGEDGQAADGSMQPTSWRQEPQLC
GMGTEQGCWIPVSSDKGSCPQVMERSFHMPSYGTQTLEGGVEKPHSLLSANPLWQQ
RALDPPHQMELTQ*
Glia1-derived neurotrophic factor (GDNF) polypeptide(sequence of
overexpressed GDNF (homo sapiens):
(SEQ ID NO: 6)
MKLWDVVAVCLVLLHTASAFPLPAGKRPPEAPAEDRSLGRRRAPFALSSDSNMPED
YPDQFDDVMDFIQATIKRLKRSPDKQMAVLPRRERNRQAAAANPENSRGKGRRGQR
GKNRGCVLTAIHLNVTDLGLGYETKEELIFRYCSGSCDAAETTYDKILKNLSRNRRLV
SDKVGQACCRPIAFDDDLSFLDDNL VYHILRKHSAKRCGCI*
Mus musculus vascular endothelial growth factor A (Vegfa)
(SEQ ID NO: 7)
MNFLLSWVHWTLALLLYLHHAKWSQAAPTTEGEQKSHEVIKFMDVYQRSYCRPIET
LVDIFQEYPDEIEYIFKPSCVPLMRCAGCCNDEALECVPTSESNITMQIMRIKPHQSQHI
GEMSFLQHSRCECRPKKDRTKPEKKSVRGKGKGQKRKRKKSRFKSWSVHCEPCSER
RKHLFVQDPQTCKCSCKNTDSRCKARQLELNERTCRCDKPRR*
Homo sapiens vascular endothelial growth factor A (VEGFA)
(SEQ ID NO: 12)
MNFLLSWVHWSLALLLYLHHAKWSQAAPMAEGGGQNHHEVVKFMDVYQRSYCHP
IETLVDIFQEYPDEIEYIFKPSCVPLMRCGGCCNDEGLECVPTEESNITMQIMRIKPHQG
QHIGEMSFLQHNKCECRPKKDRARQEKKSVRGKGKGQKRKRKKSRYKSWSVYVGA
RCCLMPWSLPGPHPCGPCSERRKHLFVQDPQTCKCSCKNTDSRCKARQLELNERTCR
CDKPRR*

In other embodiments, the polypeptides of the invention have at least 60%, 70%, 75%, 80%, 85%, 90%, 91%, 92%, 93%, 94%, 95%, 96%, 97%, 98%, 99%, or 100% sequence identity to the amino acid sequence of one or more SEQ ID NOs: 1 to 12. In certain embodiments, the polypeptides having at least 60%, 70%, 75%, 80%, 85%, 90%, 91%, 92%, 93%, 94%, 95%, 96%, 97%, 98%, or 99% identity to the amino acid sequence of one or more SEQ ID NOs: 1 to 12 contain substitutions (such as conservative substitutions), insertions, or deletions relative to the reference sequence, but still maintaining their biological activity. In certain embodiments, a total of 1 to 10 amino acids have been substituted, inserted and/or deleted in one or more SEQ ID NOs: 1 to 12, but still maintaining their biological activity. Optionally, the polypeptide sequences SEQ ID NOs: 1 to 12 include post-translational modifications of that sequences.

In further embodiments, the present invention also provides variants of polypeptides of the invention, as described herein. As used herein, the term “variant” refers to naturally occurring genetic variations and recombinantly prepared variations, each of which contain one or more changes in its amino acid sequence compared to a reference polypeptide Spp1, Igf1, Cntf, FGF2, EGF, GDNF or VEGF, such as SEQ ID NO: 1, 2, 3, 4, 5, 6, 7, 8, 9, 10, 11 or 12. Such changes include those in which one or more amino acid residues have been modified by amino acid substitution, addition or deletion. The term “variant” encompasses orthologs of human Spp1, Igf1, Cntf, FGF2, EGF, VEGF and/or GDNF, including for example mammalian Spp1, Igf1, Cntf, FGF2, EGF, VEGF and/or GDNF, such as, but not limited to Spp1, Igf1, Cntf, FGF2, EGF, VEGF and/or GDNF orthologs from a non-human primate, cat, dog, sheep, goat, horse, cow, pig, bird, and rodent such as but not limited to mouse and rat. In a non-limiting example, mouse Spp1, Igf1, Cntf, FGF2 and/or EGF, exemplified herein as amino acid sequences SEQ ID NOs: 1, 3, 4, 5 and 7 is an ortholog of human Spp1, Igf1, Cntf, FGF2 and/or EGF. In some cases, preferred variants have at least 60%, 75%, 80%, 85%, 90%, 91%, 92%, 93%, 94%, 95%, 96%, 97%, 98%, or 99% identity to SEQ ID NO: 1, 2, 3, 4, 5, 6, 7, 8, 9, 10, 11 or 12.

To determine the percent identity of two amino acid sequences or of two nucleic acid sequences, the sequences are aligned for optimal comparison purposes (e.g., gaps can be introduced in the sequence of a first amino acid or nucleic acid sequence for optimal alignment with a second amino acid or nucleic acid sequence). The amino acid residues or nucleotides at corresponding amino acid positions or nucleotide positions are then compared. When a position in the first sequence is occupied by the same amino acid residue or nucleotide as the corresponding position in the second sequence, then the molecules are identical at that position. The percent identity between the two sequences is a function of the number of identical positions shared by the sequences (i.e., % identity=number of identical overlapping positions/total number of positions×100%). In one embodiment, the two sequences are the same length. The determination of percent identity between two sequences can also be accomplished using a mathematical algorithm. A preferred, non-limiting example of a mathematical algorithm utilized for the comparison of two sequences is the algorithm of Karlin and Altschul, PNAS, 87:2264-2268 (1990), modified as in Karlin and Altschul, PNAS, 90:5873-5877 (1993). Such an algorithm is incorporated into the NBLAST and XBLAST programs of Altschul et al., J. Mol. Biol., 215:403 (1990). BLAST nucleotide searches are performed with the NBLAST nucleotide program parameters set, e.g., for score=100, wordlength=12 to obtain nucleotide sequences homologous to a nucleic acid molecule described herein. BLAST protein searches are performed with the XBLAST program parameters set, e.g., to score 50, wordlength=3 to obtain amino acid sequences homologous to a protein molecule of the present invention. To obtain gapped alignments for comparison purposes, Gapped BLAST are utilized as described in Altschul et al. (Nucleic Acids Res., 25:3389-3402 (1997)). Alternatively, PSI BLAST is used to perform an iterated search which detects distant relationships between molecules. When utilizing BLAST, Gapped BLAST, and PSI Blast programs, the default parameters of the respective programs (e.g., of XBLAST and NBLAST) are used (see, e.g., the NCBI website). Another preferred, non-limiting example of a mathematical algorithm utilized for the comparison of sequences is the algorithm of Myers and Miller (CABIOS, 4:11-17 (1988)). Such an algorithm is incorporated in the ALIGN program (version 2.0) which is part of the GCG sequence alignment software package. When utilizing the ALIGN program for comparing amino acid sequences, a PAM120 weight residue table, a gap length penalty of 12, and a gap penalty of 4 is used. The percent identity between two sequences is determined using techniques similar to those described above, with or without allowing gaps. In calculating percent identity, typically only exact matches are counted.

In certain embodiments, the nucleic acids encode biologically active fragments of polypeptides of the invention, as described herein.

The term “polypeptide”, such as osteopontin (Spp1) polypeptide, insulin-like growth factor 1 (Igf1) polypeptide, ciliary-derived neurotrophic factor (Cntf) polypeptide, fibroblast growth factor 2 (FGF2) polypeptide, epidermal growth factor (EGF) polypeptide, glial-derived neurotrophic factor (GDNF) polypeptide, vascular endothelial growth factor (VEGF), encompasses biologically active fragments thereof, such as fragments of SEQ ID Nos: 1, 2, 3, 4, 5, 6, 7, 8, 9, 10, 11 and 12 and variants thereof, operable in pharmaceutical combinations and methods described herein.

The polypeptides of the inventions and nucleic acids may be isolated from natural sources, such as the brain of an organism or cells of a cell line which expresses said polypeptides. Alternatively, polypeptides or nucleic acid may be generated recombinantly, such as by expression using an expression construct, in vitro or in vivo. Polypeptides and nucleic acids may also be synthesized by well-known methods. Polypeptides included in pharmaceutical combinations and methods described herein can be produced using recombinant nucleic acid technology. Recombinant production includes introducing a recombinant expression vector encompassing a DNA sequence encoding a polypeptide into a host cell. Thus in some embodiments, nucleic acids encoding polypeptides of the invention introduced into a host cell to produce polypeptides of the invention, can encode SEQ ID NOs: 1, 2, 3, 4, 5, 6, 7, 8, 9, 10, 11 and 12, biologically active fragments thereof, or variants thereof.

It will be appreciated that the first agent, the second agent and the third agent of the pharmaceutical combination of the invention may be administered simultaneously, either in the same formulation or different pharmaceutical formulations, separately or sequentially. Thus in one embodiment, the first agent, the second agent and the third agent are administered simultaneously. In one such embodiment, administration in combination is accomplished by combining the first agent, the second agent and the third agent in a single dosage form. In another embodiment, the first agent, the second agent and the third agent are administered sequentially or separately. In such embodiment, the first agent is first to be administered, the second agent is second to the administered and the third agent is last to be administered. In another embodiment the first agent, the second agent and the third agent are administered through the same route or through different routes. In some embodiments, the time period between administration of the first agent and administration of the second agent can be about 1 hour, 2 hours, 3 hours, 5 hours, 8 hours, 10 hours, 12 hours, 15 hours, 18 hours, 20 hours, 24 hours, 36 hours, 48 hours, 3 days, 4 days, 5 days, 6 days, 7 days, 10 days, 14 days, 21 days, 28 days, or 30 days. In some embodiments, the second agent and the third agent are administered simultaneously. In further embodiments, time period between administration of the second agent and administration of the third agent can be about 1 hour, 2 hours, 3 hours, 5 hours, 8 hours, 10 hours, 12 hours, 15 hours, 18 hours, 20 hours, 24 hours, 36 hours, 48 hours, 3 days, 4 days, 5 days, 6 days, 7 days, 10 days, 14 days, 21 days, 28 days, or 30 days. The same applies, mutatis mutandis, for a pharmaceutical combination for use in the treatment of incomplete SCI or stroke, where only the first agent and the second agent are present.

In some embodiments, the pharmaceutical combinations of the invention further comprise a pharmaceutically acceptable carrier. As used here, the term “pharmaceutically acceptable” refers to those compounds, materials, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio. As used here, the term “pharmaceutically acceptable carrier” means a pharmaceutically acceptable material, composition or vehicle, such as a liquid or solid filler, diluent, excipient, manufacturing aid (e.g., lubricant, talc magnesium, calcium or zinc stearate, or steric acid), or solvent encapsulating material, involved in carrying or transporting the subject compound from one organ, or portion of the body, to another organ, or portion of the body. Each carrier must be “acceptable” in the sense of being compatible with the other ingredients of the formulation and not injurious to the patient. Some examples of materials which can serve as pharmaceutically acceptable carriers include, but are not limited to: (1) sugars, such as lactose, glucose and sucrose; (2) starches, such as corn starch and potato starch; (3) cellulose, and its derivatives, such as sodium carboxymethyl cellulose, methylcellulose, ethyl cellulose, microcrystalline cellulose and cellulose acetate; (4) powdered tragacanth; (5) malt; (6) gelatin; (7) lubricating agents, such as magnesium stearate, sodium lauryl sulfate and talc; (8) excipients, such as cocoa butter and suppository waxes; (9) oils, such as peanut oil, cottonseed oil, safflower oil, sesame oil, olive oil, corn oil and soybean oil; (10) glycols, such as propylene glycol; (11) polyols, such as glycerin, sorbitol, mannitol and polyethylene glycol (PEG); (12) esters, such as ethyl oleate and ethyl laurate; (13) agar; (14) buffering agents, such as magnesium hydroxide and aluminium hydroxide; (15) alginic acid; (16) pyrogen-free water; (17) isotonic saline; (18) Ringer's solution; (19) ethyl alcohol; (20) pH buffered solutions; (21) polyesters, polycarbonates and/or polyanhydrides; (22) bulking agents, such as polypeptides and amino acids (23) serum component, such as serum albumin, HDL and LDL; (22) C2-C12 alcohols, such as ethanol; and (24) other non-toxic compatible substances employed in pharmaceutical formulations. Wetting agents, binding agents, fillers, lubricants, coloring agents, disintegrants, release agents, coating agents, sweetening agents, flavoring agents, perfuming agents, preservative, water, salt solutions, alcohols, antioxidants, polyethylene glycols, gelatin, lactose, amylose, magnesium stearate, talc, silicic acid, viscous paraffin, hydroxymethylcellulose, polyvinylpyrrolidone and the like can also be present in the formulation. The terms such as “excipient”, “carrier”, “pharmaceutically acceptable carrier” or the like are used interchangeably herein.

In other embodiments, the pharmaceutical combinations of the invention further comprise an agent that facilitates passage through the blood brain barrier. In one embodiment, the pharmaceutically acceptable agent facilitates the passage through, or has the capacity to pass through, the blood brain barrier.

The term “vector”, as used herein, refers to a nucleic acid construct designed for delivery to a host cell or for transfer between different host cells. In embodiments of the present invention, a vector is viral vector. The term “vector” encompasses any genetic element that is capable of replication when associated with the proper control elements and that can transfer gene sequences to cells. A vector can include, but is not limited to, a cloning vector, an expression vector, a plasmid, phage, transposon, mRNAs, cosmid, artificial chromosome, virus, virion, etc.

As used herein, the term “expression vector” refers to a vector that directs expression of an RNA or polypeptide (e.g., osteopontin (Spp1) polypeptide as set forth in SEQ ID NO: 1 or SEQ ID NO: 8, insulin-like growth factor 1 (Igf1) polypeptide as set forth in SEQ ID NO: 2, ciliary-derived neurotrophic factor (Cntf) polypeptide as set forth in SEQ ID NO: 3 or SEQ ID NO: 9, fibroblast growth factor 2 (FGF2) polypeptide as set forth in SEQ ID NO: 4 or SEQ ID NO: 10, epidermal growth factor (EGF) polypeptide as set forth in SEQ ID NO: 5 or SEQ ID NO: 11, glial-derived neurotrophic factor (GDNF) polypeptide as set forth in SEQ ID NO: 6 or vascular endothelial growth factor (VEGF) polypeptide as set forth in SEQ ID NO: 7 or SEQ ID NO: 12) from nucleic acid sequences contained therein linked to transcriptional regulatory sequences on the vector. The sequences expressed will often, but not necessarily, be heterologous to the cell. An expression vector may comprise additional elements, for example, the expression vector may have two replication systems, thus allowing it to be maintained in two organisms, for example in human cells for expression and in a prokaryotic host for cloning and amplification. The term “expression” refers to the cellular processes involved in producing RNA and proteins and as appropriate, secreting proteins, including where applicable, but not limited to, for example, transcription, transcript processing, translation and protein folding, modification and processing. “Expression products” include RNA transcribed from a gene, and polypeptides obtained by translation of mRNA transcribed from a gene. The term “gene” means the nucleic acid sequence which is transcribed (DNA) to RNA in vitro or in vivo when operably linked to appropriate regulatory sequences. The gene may or may not include regions preceding and following the coding region, e.g. 5′ untranslated (5′UTR) or “leader” sequences and 3′ UTR or “trailer” sequences, as well as intervening sequences (introns) between individual coding segments (exons).

Integrating vectors have their delivered RNA/DNA permanently incorporated into the host cell chromosomes. Non-integrating vectors remain episomal which means the nucleic acid contained therein is never integrated into the host cell chromosomes. Examples of integrating vectors include retroviral vectors, lentiviral vectors, hybrid adenoviral vectors, and herpes simplex viral vector.

One example of a non-integrative vector is a non-integrative viral vector. Non-integrative viral vectors eliminate the risks posed by integrative retroviruses, as they do not incorporate their genome into the host DNA. One example is the Epstein Barr oriP/Nuclear Antigen-1 (“EBNA1”) vector, which is capable of limited self-replication and known to function in mammalian cells. As containing two elements from Epstein-Barr virus, oriP and EBNA1, binding of the EBNA1 protein to the virus replicon region oriP maintains a relatively long-term episomal presence of plasmids in mammalian cells. This particular feature of the oriP/EBNA1 vector makes it ideal for generation of integration-free iPSCs. Another non-integrative viral vector is adenoviral vector and the adeno-associated viral (AAV) vector. Still another non-integrative viral vector is RNA Sendai viral vector, which can produce protein without entering the nucleus of an infected cell. The F-deficient Sendai virus vector remains in the cytoplasm of infected cells for a few passages, but is diluted out quickly and completely lost after several passages (e.g., 10 passages). Further example of a non-integrative vector is a minicircle vector. Minicircle vectors are circularized vectors in which the plasmid backbone has been released leaving only the eukaryotic promoter and cDNA(s) that are to be expressed.

In various embodiments, the vector crosses the blood brain barrier. In other embodiments, any agent described herein is formulated to cross the blood brain barrier. The blood brain barrier is a highly selective semipermeable membrane barrier that separates the circulating blood from the brain extracellular fluid in the central nervous system (CNS). For therapeutics needed to be delivered to the CNS, a skilled clinician can directly deliver a therapeutic, such as agents of the invention or the pharmaceutical combinations of the invention, to the spinal canal. For direct administration into the spinal canal, the agents and the pharmaceutical combinations described herein will be administered via intrathecal administration by a skilled clinician. Intrathecal administration is a route of drug administration in which the drug is directly injected in the spinal canal or in the subarachnoid space, allowing it to directly reach the cerebrospinal fluid (CSF). A vector can be packaged with at least a second agent that permeabilizes the blood brain barrier. One skilled in the art can determine if a vector has crossed the blood brain barrier, e.g., by determining if the vector is detected in, e.g., spinal fluid, following administration.

In some embodiments, a vector is a messenger RNA (mRNA). In recent years, messenger RNA therapy has become an increasingly important option for treatment of various diseases and conditions, in particular, for those associated with deficiency of one or more proteins. The term “messenger RNA (mRNA)” refers to a polynucleotide that encodes at least one polypeptide. mRNA as used herein encompasses both modified and unmodified RNA. mRNA may contain one or more coding and non-coding regions. mRNA can be purified from natural sources, produced using recombinant expression systems and optionally purified, chemically synthesized, etc. Where appropriate, e.g., in the case of chemically synthesized molecules, mRNA can comprise nucleoside analogues such as analogues having chemically modified bases or sugars, backbone modifications, etc. An mRNA sequence is presented in the 5′ to 3′ direction unless otherwise indicated. In some embodiments, an mRNA is or comprises natural nucleosides (e.g., adenosine, guanosine, cytidine, uridine); nucleoside analogues (e.g., 2-aminoadenosine, 2-thiothymidine, inosine, pyrrolo-pyrimidine, 3-methyl adenosine, 5-methylcytidine, C-5 propynyl-cytidine, C-5 propynyl-uridine, 2-aminoadenosine, C5-bromouridine, C5-fluorouridine, C5-iodouridine, C5-propynyl-uridine, C5-propynyl-cytidine, C5-methylcytidine, 2-aminoadenosine, 7-deazaadenosine, 7-deazaguanosine, 8-oxoadenosine, 8-oxoguanosine, O(6)-methylguanine, and 2-thiocytidine); chemically modified bases; biologically modified bases (e.g., methylated bases); intercalated bases; modified sugars (e.g., 2′-fluororibose, ribose, 2′-deoxyribose, arabinose, and hexose); modified phosphate groups (e.g., phosphorothioates and 5′-N-phosphoramidite linkages), as well as any combination of the foregoing. An mRNA according to the invention may comprises one or more modified nucleotides for instance to enhance its stability or to avoid or reduce its degradation upon delivery into a subject.

Typically, mRNA synthesis includes the addition of a “cap” on the N-terminal (5′) end, and a “tail” on the C-terminal (3′) end. The presence of the cap is important in providing resistance to nucleases found in most eukaryotic cells. The presence of a “tail” serves to protect the mRNA from exonuclease degradation.

In some embodiments, the mRNA is delivered to neurons located within the brain. In some embodiments, the mRNA is delivered to neurons located within the spinal cord. In some embodiments, the mRNA is delivered to motor neurons. In some embodiments, the mRNA is delivered to upper motor neurons and/or lower motor neurons.

In some embodiments, the mRNA is administered intrathecally to a subject, such that the administering of the composition results in the intracellular delivery of mRNA in neurons in the brain and/or spinal cord. Intracellular delivery of the mRNA results in expression of the protein encoded by the mRNA. In some embodiments, the encoded protein is expressed within the cytosol of the neurons. In some embodiments, the encoded protein is expressed and secreted extracellularly form the neurons after expression.

mRNA may be delivered to the CNS as naked RNA (unpackaged) or via delivery vehicles. As used herein, the terms “delivery vehicle,” “transfer vehicle,” “Nanoparticle” or grammatical equivalent, are used interchangeably.

In some embodiments, mRNAs may be delivered via a single delivery vehicle. In some embodiments, mRNAs may be delivered via one or more delivery vehicles each of a different composition. According to various embodiments, suitable delivery vehicles include, but are not limited to, polymer based carriers, such as polyethyleneimine (PEI), lipid nanoparticles and liposomes, nanoliposomes, ceramide-containing nanoliposomes, proteoliposomes, both natural and synthetically-derived exosomes, natural, synthetic and semi-synthetic lamellar bodies, nanoparticulates, calcium phosphor-silicate nanoparticulates, calcium phosphate nanoparticulates, silicon dioxide nanoparticulates, nanocrystalline particulates, semiconductor nanoparticulates, poly(D-arginine), sol-gels, nanodendrimers, starch-based delivery systems, micelles, emulsions, niosomes, multi-domain-block polymers (vinyl polymers, polypropyl acrylic acid polymers, dynamic polyconjugates), dry powder formulations, plasmids, viruses, calcium phosphate nucleotides, aptamers, peptides and other vectorial tags.

In some embodiments, a therapeutically effective dose of mRNA may range for instance from about 0.001 mg/kg body weight to 10 mg/kg body weight, from about 0.005 mg/kg body weight to 10 mg/kg body weight, from about 0.01 mg/kg body weight to 10 mg/kg body weight, from about 0.01 mg/kg body weight to 9 mg/kg body weight, from about 0.01 mg/kg body weight to 8 mg/kg body weight, from about 0.01 mg/kg body weight to 7 mg/kg body weight, from about 0.01 mg/kg body weight to 6 mg/kg body weight, from about 0.01 mg/kg body weight to 5 mg/kg body weight, from about 0.01 mg/kg body weight to 4 mg/kg body weight, from about 0.01 mg/kg body weight to 3 mg/kg body weight, from about 0.01 mg/kg body weight to 2 mg/kg body weight, from about 0.01 mg/kg body weight to 1 mg/kg body weight, from about 0.01 mg/kg body weight to 0.5 mg/kg body weight, from about 0.1 mg/kg body weight to 10 mg/kg body weight, from about 0.1 mg/kg body weight to 5 mg/kg body weight, from about 0.5 mg/kg body weight to 10 mg/kg body weight, or from about 0.5 mg/kg body weight to 5 mg/kg body weight.

It has been in fact surprisingly demonstrated by the present inventors that not only a combination of the first, second and third agent according to the invention is advantageous for the functional and anatomical recovery of complete SCI, but that additionally the administration in a subject of only said first agent, or only said second agent or a combination thereof has remarkable effects and functional benefits in less severe injuries, such as neuronal damage after a stroke, and enables improved recovery of walking after incomplete SCI. Sustained expression of exogenous nucleic acids of the various agents, alone or in combination, also resulted important to attain the spinal cord repair, axon regeneration and functional improvement of limb movements.

Thus another aspect of the present invention provides a pharmaceutical combination of the invention for use in a method of treating an incomplete spinal cord injury or a stroke in a subject.

According to this aspect of the invention, the pharmaceutical combination comprises the combination of the first agent and the second agent, or the first agent alone, or the second agent alone.

Another aspect of the present invention provides a method of treating an incomplete spinal cord injury or a stroke in a subject comprising administering to the subject the pharmaceutical combination of the invention comprising the first agent, the second agent or the combination thereof.

In preferred embodiments of the method of treating an incomplete spinal cord injury or a stroke, the pharmaceutical combination of the invention or the first agent and/or the second agent are administered after the incomplete spinal cord or the stroke.

Another aspect of the present invention provides a pharmaceutical combination of the invention for use in a method of treating a complete spinal cord injury in a subject, wherein the pharmaceutical combination comprises the first agent, the second agent and the third agent.

Another aspect of the present invention provides a method of treating a complete spinal cord injury in a subject comprising administering to the subject the pharmaceutical combination of the invention comprising the first agent, the second agent or the combination thereof.

In preferred embodiments of the method of treating a complete spinal cord injury, the pharmaceutical combination of the invention or the first, second and third agents of the invention are administered after the complete spinal cord injury.

According to some embodiments of the pharmaceutical combinations for use of the invention and the methods of treating of the invention, said method of treating further comprises an initial assessment step of determining whether the subject suffers from an incomplete spinal cord injury, a complete spinal cord injury or a stroke. In an embodiment, the initial assessment step comprises analysis of spinal cord injury symptoms or stroke symptoms selected from the group comprising one or more of loss or impairment of motor function(s), loss or impairment of sensory function(s) and loss or impairment of autonomic function(s), in the cervical, thoracic, lumbar or sacral segments of the spinal cord, and/or the brain.

Another aspect of the present invention provides a method of treating neurotrauma in a subject, the method comprising:
administering to a subject in need thereof one of
   a first agent, or
   a second agent, or
   a pharmaceutical combination of said first agent and said second agent,
if the neurotrauma is incomplete spinal cord injury or stroke,
   or
administering to a subject in need thereof a pharmaceutical combination comprising a first agent, a second agent and a third agent if the neurotrauma is complete spinal cord injury,
wherein
   said first agent comprises exogenous nucleic acid encoding an osteopontin (Spp1) polypeptide as set forth in SEQ ID NO: 1 or SEQ ID NO: 8, insulin-like growth factor 1 (Igf1) polypeptide as set forth in SEQ ID NO: 2 and ciliary-derived neurotrophic factor (Cntf) polypeptide as set forth in SEQ ID NO: 3 or SEQ ID NO: 9;
   said second agent comprises exogenous nucleic acid encoding a fibroblast growth factor 2 (FGF2) polypeptide as set forth in SEQ ID NO: 4 or SEQ ID NO: 10, epidermal growth factor (EGF) polypeptide as set forth in SEQ ID NO: 5 or SEQ ID NO: 11, and a glial-derived neurotrophic factor (GDNF) polypeptide as set forth in SEQ ID NO: 6;
   said third agent comprises exogenous nucleic acid encoding a glial-derived neurotrophic factor (GDNF) polypeptide as set forth in SEQ ID NO: 6;
and wherein
   a) the first agent, the second agent and the third agent provide sustained expression and secretion of effective amounts of the polypeptides during at least 3 days or more, and
   b) when in combination, said agents are administered sequentially or simultaneously.

In preferred embodiments of the method of treating neurotrauma, the pharmaceutical combination of the invention or the first agent, the second agent and the third agent (depending of the injury) are administered after the incomplete spinal cord injury, the stroke or the complete spinal cord injury.

Advantageously, in some embodiments the second agent further comprises exogenous nucleic acid encoding a vascular endothelial growth factor (VEGF) polypeptide as set forth in SEQ ID NO: 7 or SEQ ID NO: 12.

In some embodiments, the method of treating of the invention further comprises an initial assessment step of determining whether the neurotrauma is incomplete spinal cord injury, complete spinal cord injury or stroke. In an embodiment, the initial assessment step comprises analysis of spinal cord injury symptoms or stroke symptoms selected from the group comprising one or more of loss or impairment of motor function(s), loss or impairment of sensory function(s) and loss or impairment of autonomic function(s), in the cervical, thoracic, lumbar or sacral segments of the spinal cord, and/or the brain.

According to an embodiment of the invention, the complete or incomplete spinal cord injury is the spinal cord injury of the cervical, thoracic, and/or lumbar spinal cord.

According to another embodiment of the invention, the treatment of stroke is neuronal, functional and anatomical repair of brain after a stroke.

In an embodiment, the present invention provides the ability of transected axons to form functional connections with their natural target regions in the spinal cord to improve walking after anatomically complete spinal cord injury (SCI). According to this embodiment, a method of treating a complete spinal cord injury comprises the administration of lentivirus vectors (LV) at three different locations. (1) Rostral to the spinal cord injury, a combination (cocktail) of three lentivirus vectors is administered, wherein each lentivirus vector respectively expressing one different key growth factor that upregulates dormant developmental growth programs selected from Igf1, Cntf, and Spp1 (as disclosed herein). (2) In the spinal cord injury, a combination (cocktail) of three lentivirus vectors is administered, wherein two lentivirus vectors respectively expressing different growth factors that manipulate the injury microenvironment selected from FGF2 and EGF (as disclosed herein) and a third lentivirus vector expressing factor that activates chemoattraction, i.e. GDNF (as disclosed herein). (3) Caudal to the spinal cord injury, a lentivirus vector expressing GDNF (as disclosed herein) is administered. According to this embodiment, replacing adenovirus vectors (AAVs) with combinations of individual growth factor expressing lentivirus vectors (LVs) enables better treatment of a complete spinal cord injury. This individual delivery of Igf1, Cntf, Spp1, FGF2, EGF and GDNF at three different locations of a spinal cord injury led to robust regeneration of axons through an anatomically complete spinal cord injury (SCI). The consequence of this regeneration was the gradual recovery of walking.

Thus according to another embodiment, the present invention provides a method of treating a complete spinal cord injury in a subject, comprising the steps of
i. administering rostral to the spinal cord injury a first agent consisting of
   a. a lentivirus vector comprising exogenous nucleic acid encoding an osteopontin (Spp1) polypeptide as set forth in SEQ ID NO: 1 or SEQ ID NO: 8,
   b. a lentivirus vector comprising exogenous nucleic acid encoding insulin-like growth factor 1 (Igf1) polypeptide as set forth in SEQ ID NO: 2, and
   c. a lentivirus vector comprising exogenous nucleic acid encoding ciliary-derived neurotrophic factor (Cntf) polypeptide as set forth in SEQ ID NO: 3 or SEQ ID NO: 9;
ii. administering in the spinal cord injury a second agent consisting of
   a. a lentivirus vector comprising exogenous nucleic acid encoding a fibroblast growth factor 2 (FGF2) polypeptide as set forth in SEQ ID NO: 4 or SEQ ID NO: 10,
   b. a lentivirus vector comprising exogenous nucleic acid encoding epidermal growth factor (EGF) polypeptide as set forth in SEQ ID NO: 5 or SEQ ID NO: 11, and
   c. a lentivirus vector comprising exogenous nucleic acid encoding a glial-derived neurotrophic factor (GDNF) polypeptide as set forth in SEQ ID NO: 6; and
iii. administering caudal to the spinal cord injury a third agent consisting of a lentivirus vector comprising exogenous nucleic acid encoding a glial-derived neurotrophic factor (GDNF) polypeptide as set forth in SEQ ID NO: 6.

In preferred embodiments of the method of treating a complete spinal cord injury, the first agent, the second agent and the third agent are administered after the complete spinal cord injury.

According to an embodiment of the invention, the method of treating comprises sequential or simultaneous administration of the first agent, the second agent, and the third agent according to the invention for spinal cord repair of the cervical, thoracic, and/or lumbar spinal cord by targeting neurons expressing Vsx2 and/or Zfhx3 (projection V2a neurons). Sequential or simultaneous administration of the first agent, the second agent, and the third agent according to the invention is foreseen for spinal cord repair of the cervical, thoracic, and/or lumbar spinal cord following a complete SCI.

According to an embodiment of the method of the invention, the method comprises sequential or simultaneous administration of the first agent and/or the second agent according to the invention for spinal cord repair of the cervical, thoracic, and/or lumbar spinal cord, and/or for brain neuronal functional regeneration, by targeting V1 neurons, V0 neurons, V2b neurons, CSF contacting neurons, Vglut2 neurons, Vgat neurons, Chat neurons, Vsx2 and/or Zfhx3 neurons. Sequential or simultaneous administration of the first agent and/or the second agent according to the invention is foreseen for spinal cord repair of the cervical, thoracic, and/or lumbar spinal cord following an incomplete SCI, and/or for brain neuronal functional regeneration following a stroke.

According to an embodiment of the invention, the spinal cord repair of the cervical, thoracic, and/or lumbar spinal cord and/or brain neuronal functional and anatomical repair is obtained by targeting V1 neurons, V0 neurons, V2b neurons, CSF contacting neurons, Vglut2 neurons, Vgat neurons, Chat neurons, Vsx2 and/or Zfhx3 (projection V2a neurons).

According to an embodiment of the method of the invention, the spinal cord repair consists of axon regeneration and restoring neurological functions.

According to an embodiment of the method of the invention, the spinal cord repair consists of blood-brain-barrier and astrocyte barrier re-formation and restoring neurological functions.

According to an embodiment of the method of the invention, the spinal cord injury symptoms or stroke symptoms are selected from the group comprising one or more of loss or impairment of motor function(s), loss or impairment of sensory function(s) and loss or impairment of autonomic function(s), in the cervical, thoracic, lumbar or sacral segments of the spinal cord, and/or the central nervous system in general, and brain in particular.

Another aspect of the present invention provides a method for regenerating spinal cord neurons within a subject having a complete or incomplete spinal cord injury (SCI), wherein said method comprises administering a pharmaceutical combination of the invention in the spinal cord and wherein said spinal cord neurons are one or more populations of neurons present in the spinal cord.

Another aspect of the present invention provides a method for regenerating brain neurons within a subject having survived a stroke, wherein said method comprises administering the pharmaceutical combination of the invention in the brain, and wherein said brain neurons are one or more populations of brain neurons.

According to an embodiment of the method of the invention, the spinal cord neurons are dorsal, medial and/or ventral.

According to an embodiment of the method of the invention, the spinal cord and brain neurons are V1 neurons, V0 neurons, V2b neurons, CSF contacting neurons, Vglut2 neurons, Vgat neurons, Chat neurons, Vsx2 and Zfhx3 neurons.

According to an embodiment of the method for spinal cord repair of the invention and the method for regenerating spinal cord or brain neurons of the invention, the pharmaceutical combination of the invention is administered by injection into the cervical, thoracic, lumbar, or sacral spinal cord of a subject, or in the brain of a subject, via methods such as stereotaxic injection, or by intravenous infusion or intravenous injection.

According to an embodiment of the method for spinal cord repair of the invention and the method for regenerating spinal cord neurons of the invention, the stereotaxic injection is guided by localisation of the spinal cord injury epicentre with methods selected from ultrasound guidance, electrophysiology, or molecular guidance.

According to an embodiment of the method for spinal cord repair of the invention and the method for regenerating spinal cord or brain neurons of the invention, the subject is a mammal, preferably a human.

In some embodiments, an effective amount of the first agent, the second agent and the third agent described herein and/or the pharmaceutical combinations, described herein, where applicable and suitable according to the present disclosure, are administered to a subject.

The term “effective amount” as used herein refers to the amount of the pharmaceutical compositions or combinations, where applicable and suitable according to the present disclosure, of the first agent, the second agent and the third agent that can be administered to a subject having or having been diagnosed to have a spinal cord injury or stroke needed to alleviate at least one or more symptom of a spinal cord injury or stroke. The term “therapeutically effective amount” therefore refers to an amount of the pharmaceutical compositions or combinations, where applicable and suitable according to the present disclosure, of the first agent, the second agent and the third agent that is sufficient to provide a particular anti-spinal cord injury or anti-stroke effect when administered to a typical subject. An effective amount as used herein, in various contexts, would also include an amount of the pharmaceutical compositions or combinations, where applicable and suitable according to the present disclosure, of the first agent, the second agent and the third agent sufficient to delay the development of a symptom of a spinal cord injury or stroke, alter the course of a symptom of a spinal cord injury or stroke (e.g., slowing the progression of loss of feeling or mobility in limbs), or reverse a symptom of a spinal cord injury or stroke (e.g., restoring feeling or mobility in limbs that was previously reduced or lost). Thus, it is not generally practicable to specify an exact “effective amount”. However, for any given case, an appropriate “effective amount” can be determined by one of ordinary skill in the art using only routine experimentation.

In one embodiment, the pharmaceutical combinations, where applicable and suitable according to the present disclosure, described herein of the first agent, the second agent, and the third agent are administered within at least 1 minute, at least 2 minutes, at least 3 minutes, at least 4 minutes, at least 5 minutes, at least 10 minutes, at least 15 minutes, at least 20 minutes, at least 25 minutes, at least 30 minutes, at least 35 minutes, at least 40 minutes, at least 45 minutes, at least 50 minutes, at least 55 minutes, at least 1 hour, at least 2 hours, at least 3 hours, at least 4 hours, at least 5 hours, at least 6 hours, at least 12 hours, at least 18 hours, at least 24 hours, at least 36 hours, at least 48 hours, at least 60 hours, at least 72 hours, at least 96 hours, at least 5 days, at least 6 days, at least 1 week, at least 2 weeks, at least 3 weeks, at least 1 month, at least 2 months, at least 3 months, at least 4 months, at least 5 months, at least 6 months, at least 7 months, at least 8 months, at least 9 months, at least 10 months, at least 11 months, at least 12 months, at least 2 years, at least 3 years, at least 4 years, or at least 5 years or more following the occurrence of the spinal cord injury or stroke.

Effective amounts, toxicity, and therapeutic efficacy can be evaluated by standard pharmaceutical procedures in cell cultures or experimental animals. The dosage can vary depending upon the dosage form employed and the route of administration utilized. The dose ratio between toxic and therapeutic effects is the therapeutic index and can be expressed as the ratio LD50/ED50. Compositions and methods that exhibit large therapeutic indices are preferred. A therapeutically effective dose can be estimated initially from cell culture assays. Also, a dose can be formulated in animal models to achieve a circulating plasma concentration range that includes the IC50 (i.e., the concentration of the agent, which achieves a half-maximal inhibition of symptoms) as determined in cell culture, or in an appropriate animal model. Levels in plasma can be measured, for example, by high performance liquid chromatography. The effects of any particular dosage can be monitored by a suitable bioassay, e.g., measuring.

The result of the method for spinal cord repair described herein as well as the method for regenerating spinal cord or brain neurons described herein is the restoration of walking or other neurological functions. Further, the advantage of the pharmaceutical combinations described herein is that it guides neurons to their proper location, delivers all growth factors, instead of the use of biomaterial depots, and improves growth factor expression and activate intrinsic neuronal growth capacity.

Those skilled in the art will appreciate that the invention described herein is susceptible to variations and modifications other than those specifically described. It is to be understood that the invention includes all such variations and modifications without departing from the spirit or essential characteristics thereof. The invention also includes all of the steps, features, compositions and compounds referred to or indicated in this specification, individually or collectively, and any and all combinations or any two or more of said steps or features. The present disclosure is therefore to be considered as in all aspects illustrated and not restrictive, the scope of the invention being indicated by the appended claims, and all changes which come within the meaning and range of equivalency are intended to be embraced therein.

The foregoing description will be more fully understood with reference to the following Examples. Such Examples, are, however, exemplary of methods of practising the present invention and are not intended to limit the application and the scope of the invention.

### Examples

### The Neurons Involved in Natural Repair

It was first aimed to determine the transcriptional identity of the neuronal subpopulations involved in the recovery of walking through natural repair mechanisms after severe but incomplete SCI. Recovery of walking occurs naturally in humans and animal models after unilateral hemisections (Brown-Séquard syndrome) that deprive walking execution centres on one side of the spinal cord from essential supraspinal inputs. Some previous studies showed that neurons located in midthoracic spinal segments can relay supraspinal commands past lateral hemisections to restore walking. Even after temporally and spatially separated hemisection lesions that interrupt all direct projections from the brain to walking execution centres, these neurons are still able to relay sufficient supraspinal input to restore voluntary unassisted walking. Ablation of these neurons did not alter walking in the absence of injury, but completely eliminated the natural recovery of walking observed after lateral hemisections. It was therefore aimed to uncover the molecular and anatomical properties of the neuronal subpopulations underlying this recovery.

To identify the neuronal subpopulations with projections to walking execution centres, rAAV2 encoding eGFP fused to the nuclear envelope protein KASH was injected into the lumbar spinal cord of uninjured mice (data not shown). This strategy labelled the nuclei of neurons with direct projections to the walking execution centres throughout the central nervous system, including relay neurons in the midthoracic spinal cord (data not shown). This strategy enabled fluorescence-activated nuclei sorting coupled to single-nucleus RNA sequencing (snRNA-seq) of projection-specific neuronal subpopulations (data not shown).

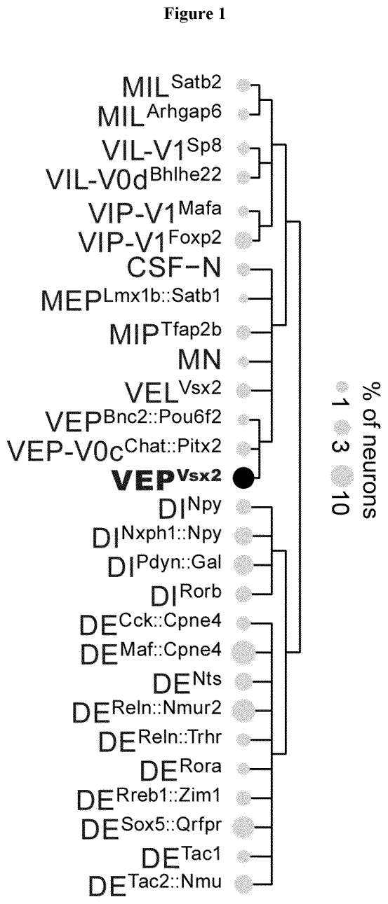

High-quality single-nucleus transcriptional profiles from 122 eGFP^{ON} and 2,823 eGFPOFF nuclei were obtained (data not shown). Unsupervised clustering identified all of the major cell types of the mouse spinal cord (data not shown). The neurons were subjected to a second round of clustering, which identified 28 subpopulations of neurons expressing canonical marker genes (FIG. 1). The taxonomy parcellated cardinal spinal neuron classes into motor-sensory, local-long range, and excitatory-inhibitory neuronal subpopulations (data not shown). Strikingly, eGFP^{ON} neurons were primarily found within a single ventral neuronal subpopulation expressing the marker Vsx2 and the key marker of long-distance projection neurons, Zfhx3 (spinal cord (SC)^{Vsx2::Zfhx3→lumba} neurons) (data not shown).

Because thoracic neurons become essential to walk after severe incomplete SCI but not in the absence of injury, it was investigated whether SC^{Vsx2::Zfhx3→lumbar} neurons are also transcriptionally perturbed following natural spinal cord repair. Neuronal nuclei from uninjured mice and mice that had recovered walking (data not shown) after temporally and spatially separated lateral hemisection SCIs were compared (data not shown). High-quality transcriptional profiles were obtained from 9,264 nuclei (data not shown), representing all the major cell types in the mouse spinal cord (data not shown). From these, the neurons were subjected to a second round of clustering. The data were integrated with the projection-specific snRNA-seq experiment, wherein the same 28 neuronal subpopulations were identified and evaluated (data not shown). Cell type prioritization revealed that SC^{Vsx2::Zfhx3→lumbar} neurons exhibited the most profound transcriptional perturbation across all neuronal subpopulations embedded in the thoracic segments of mice that had recovered walking, consistent with an involvement in natural repair (data not shown).

Anatomical features of SC^{Vsx2::Zfhx3→lumbar} neurons

The obtained results thus far implied that SC^{Vsx2::Zfhx3→lumbar} neurons are the putative neurons that restore walking after natural spinal cord repair. Therefore, it was hypothesised that these neurons must possess anatomical features compatible with the requirements to walk after paralysis.

Visualisation of the projectome from neurons embedded in the midthoracic spinal cord revealed dense projections throughout the lumbar spinal cord wherein walking execution centres reside (data not shown). To identify neuronal subpopulations possessing this projectome combined with a transcriptional phenotype consistent with prioritised neuronal subpopulations (data not shown), the distribution and connectome of Vsx2^{ON} neurons using intersectional genetics and viral tracing in Vsx2^{Cre} mice was compared. It was found that Vsx2^{ON} neurons located in the midthoracic spinal cord accounted for 5.9% of neurons in this region (data not shown), which agreed with the distribution of neurons identified in our snRNA-seq data (data not shown).

Tracing of midthoracic Vsx2^{ON} neurons revealed the expected presence of dense projections throughout walking execution centres (data not shown).

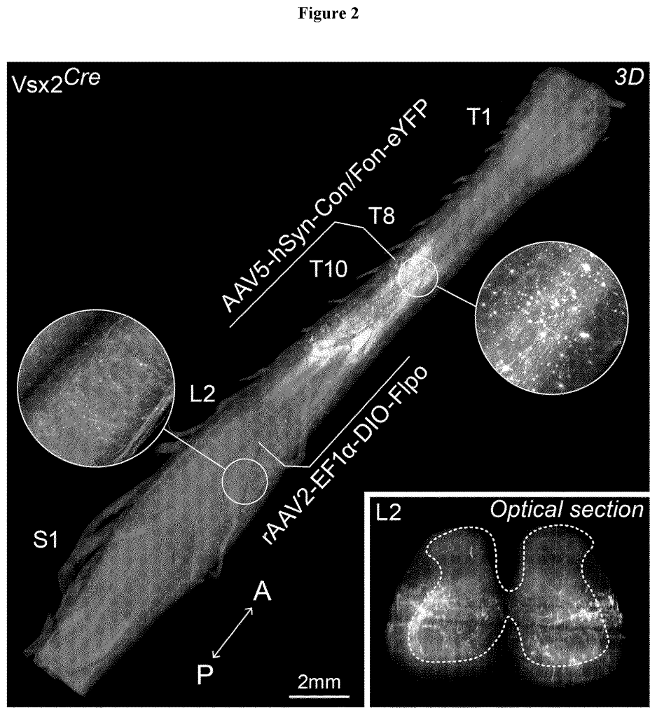

It was then analysed whether the population of midthoracic Vsx2^{ON} neurons could be stratified into subpopulations projecting locally versus over long distances. In the spinal cord, neurons with local versus long distance projections can be differentiated by the expression of Zfhx3 (data not shown). To label long-distance projecting Vsx2^{ON} neurons, rAAV2-Ef1α-DIO-Flpo was infused into the lumbar spinal cord of Vsx2^{Cre} mice followed by injections of AAV5-Con/Fon-eYFP into the thoracic spinal cord (FIG. 2). This intersectional tracing strategy enabled the exclusive labelling of Vsx2^{ON} neurons that projected to walking execution centres (FIG. 2). It was found that Zfhx3 and Vsx2 co-localized only in neurons projecting to this region (SC^{Vsx2::Zfhx3→lumbar}) (data not shown), confirming that Zfhx3 is an accurate marker to target spinal cord neuron subpopulations with long-distance projections. Quantification of local (Vsx2^{ON}Zfhx3^{OFF}) versus long-distance projecting (Vsx2^{ON} Zfhx3^{ON}) Vsx2^{ON} neurons revealed a near equal distribution of these two subpopulations throughout the midthoracic spinal cord (data not shown). Together these findings confirmed that a subset of Vsx2^{ON} neurons embedded in the midthoracic spinal cord coexpress Zfhx3 and extend dense projections to the lumbar spinal cord wherein walking execution centres reside.

It was then reasoned that in order to function as relays of supraspinal commands, S^{Vsx2::Zfhx3→lumbar} neurons must also receive direct projections from key neurons involved in the recovery of walking after paralysis. To expose this connectome, AAV5-CAG-gComet was infused into the ventral gigantocellular nucleus (vGi) of Vsx2^{Cre::tdTomato} mice, since vGi neurons are essential for this recovery (data not shown). It was found that Vsx2^{ON} Zfhx3^{ON} neurons located in the midthoracic spinal cord receive dense projections from the vGi (data not shown).

Together, these results indicated that amongst the diverse populations of cells in the midthoracic spinal cord, SC^{Vsx2::Zfhx3→lumbar} neurons were the most perturbed neuronal subpopulation during natural repair, and exhibited the relevant projectome and receptome profiles to relay supraspinal commands that mediate the recovery of walking after incomplete SCI.

Regenerating SC^{Vsx2::Zfhx3→lumbar} Neurons to their Natural Target after Complete SCI

Previous studies provided that factors essential for axon growth during development were able to support spinal cord axon regeneration across anatomically complete SCI lesions into viable neural tissue located a segment below the injury but that this regrowth did not restore walking. Based on the findings above, it was considered that restoring walking after anatomically complete SCI would require recapitulating projections that restore walking after anatomically incomplete SCI, and therefore, regrow SC^{Vsx2::Zfhx3→lumbar} neurons to their natural targets within walking execution centres.

To test this concept, firstly the intrinsic growth capacity of neurons located above the SCI was reactivated with viral overexpression of osteopontin (Spp1), insulin-like growth factor 1 (Igf1) and ciliary-derived neurotrophic factor (Cntf) (AAV-OIC). Second, the formation of axon growth supportive substrates was induced by temporal delivery of fibroblast growth factor 2 (FGF2) and epidermal growth factor (EGF). Third, biomaterial depots of glial-derived neurotrophic factor (GDNF) as a chemoattractive agent was delivered within and to sequentially spaced sites below the injury. Indeed, snRNA-seq data revealed the expression of Gdnf receptor, Gfra1, and Ret in SC^{Vsx2::Zfhx3→lumbar} neurons, both of which are required for appropriate Gdnf signalling, and immunohistochemistry of Vsx2^{ON} axons traced with AAV5-Con/Fon-eYFP further validated expression of the Gdnf receptor within the soma and along the entire length of axons traced with AAV5-Con/Fon-eYFP (data not shown).

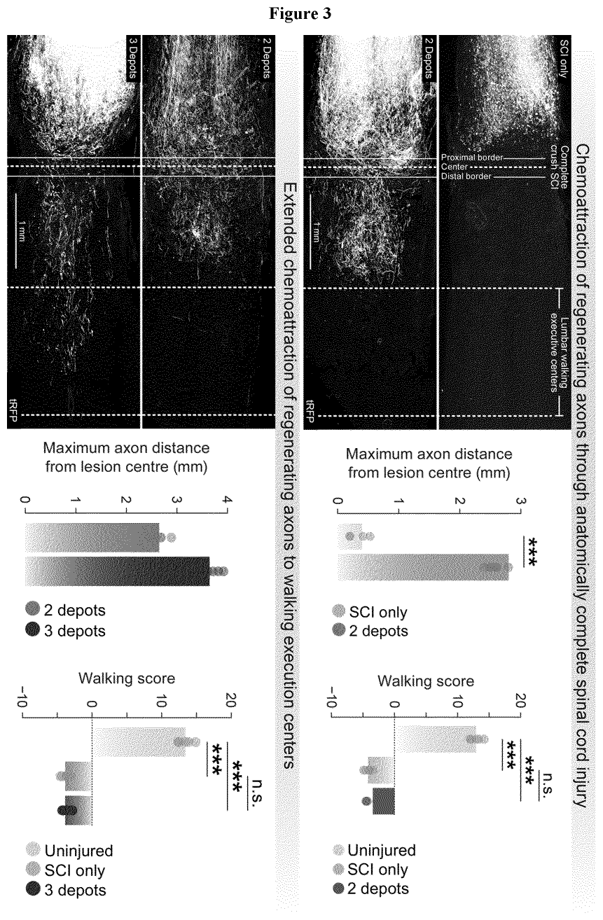

Consistent with the previous observations, it was again found that stimulated, supported, and chemoattracted axons regrew robustly through astrocyte borders, across the fibrotic scar, and into viable neural tissue below an anatomically complete SCI (FIG. 3). Nevertheless, when the regenerating axons terminated only one segment below the injury where the most distal GDNF containing biomaterial depot had been infused, even high-precision behavioural assessments conducted at 4 weeks post-injury failed to detect any recovery (data not shown). This observation contrasted with the pronounced recovery of walking observed by 4 weeks after natural spinal cord repair involving SC^{Vsx2::Zfhx3→lumbar} neurons whose axons terminated within walking execution centres located several segments more distally (data not shown).

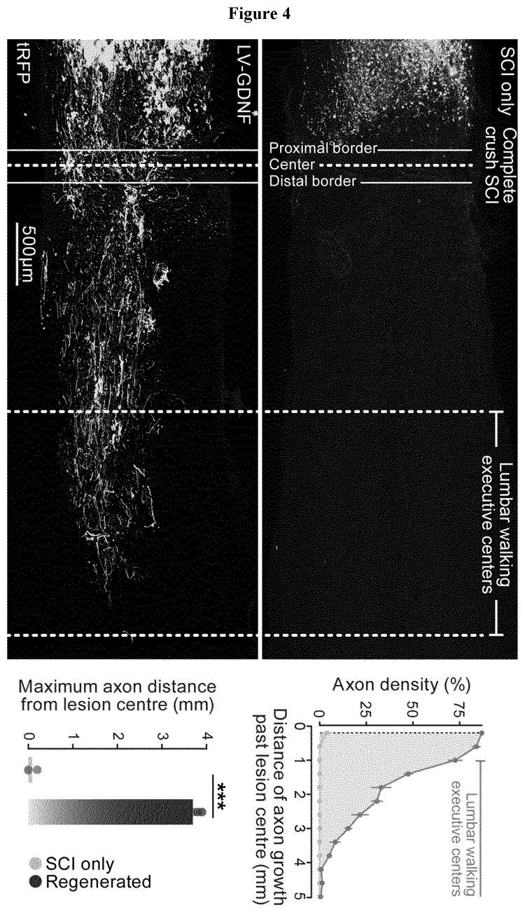

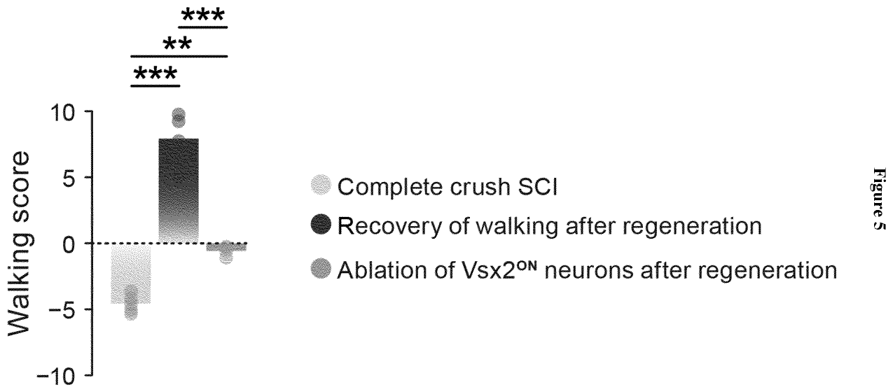

It was therefore reasoned that to recover walking after anatomically complete SCI, regenerated axons must recapitulate the topology of natural spinal cord repair and reach the lumbar spinal cord. To achieve such long-distance regeneration, an additional depot of chemoattractive GDNF was placed into the lumbar spinal cord (data not shown). However, this additional depot attracted comparatively few axons to the targeted lumbar region (data not shown), and high-precision behavioural assessments again failed to detect any recovery of walking (data not shown). It was then reasoned that the relatively slow time course of long-distance axon growth, maturation, and synapse formation might require a more sustained and higher concentration of chemoattractive growth factor delivery than was provided by the biomaterial depot. To achieve this, a lentivirus was engineered to provide sustained Gdnf delivery. Replacing biomaterial depots with lentivirus-mediated Gdnf expression enabled a remarkable regrowth of axons to their natural topological target (FIG. 4), further demonstrating that appropriate chemoattraction gradients can guide long-distance axon regeneration in a manner similar to development.

To determine whether regenerated axons originated from SC^{Vsx2::Zfhx3→lumbar} neurons, rAAV2-hSyn-KASH-eGFP was infused into the lumbar spinal cord to retrogradely express eGFP exclusively in neurons with axons that had regrown sufficiently to reach walking execution centres (data not shown). Nuclei of eGFP^{ON} neurons located above the injury were sorted for snRNAseq and the resulting single-neuron transcriptional profiles were integrated into the atlas of global cell types and neuronal subpopulations of the thoracic spinal cord (data not shown). Comparing the distribution of eGFP^{ON} neurons to the distribution of neuronal subpopulations in the uninjured spinal cord showed that SC^{Vsx2::Zfhx3→lumbar} neurons were the main virally labelled subpopulation, confirming the successful regeneration of this specific neuronal subpopulation to its natural topological target (data not shown). Retrograde tracing coupled with immunohistochemistry of Vsx2 confirmed these results (data not shown).

Finally, it was considered whether supraspinal commands could be detected below the anatomically complete SCI. It was found that microstimulation of the vGi induced large motor evoked potentials in lower limb muscles, revealing that supraspinal centres had regained access to walking execution centres (data not shown).

These results demonstrate that SC^{Vsx2::Zfhx3→lumbar} neurons can be engineered to regrow electrophysiologically active axons to their natural topological target region in the lumbar spinal cord, wherein walking execution centres reside.

### Walking Restored by Mechanism-Based Repair of Anatomically Complete SCI

Longitudinal quantification of whole-body kinematics during walking was performed in three separate cohorts of mice that underwent anatomically complete SCI in combination with targeted regrowth of SC^{Vsx2::Zfhx3→lumbar} neurons to the lumbar spinal cord. These evaluations showed that the complete crush SCI abolished walking in every mouse studied, such that even at four weeks after SCI, no mice exhibited any sign of recovery (data not shown). In fully treated mice, progressive recovery of walking emerged approximately 3 to 4 weeks after SCI (data not shown). Final evaluations were performed at 8 weeks. In cohort one, 5 out of 6 mice with anatomically complete SCI and full treatment displayed gait patterns that resembled those quantified in mice with natural spinal cord repair after incomplete SCI (data not shown). These experiments were repeated in two subsequent cohorts, with a further 13 out of 15 mice (or a total of 18 out of 21) demonstrating similar results (data not shown).

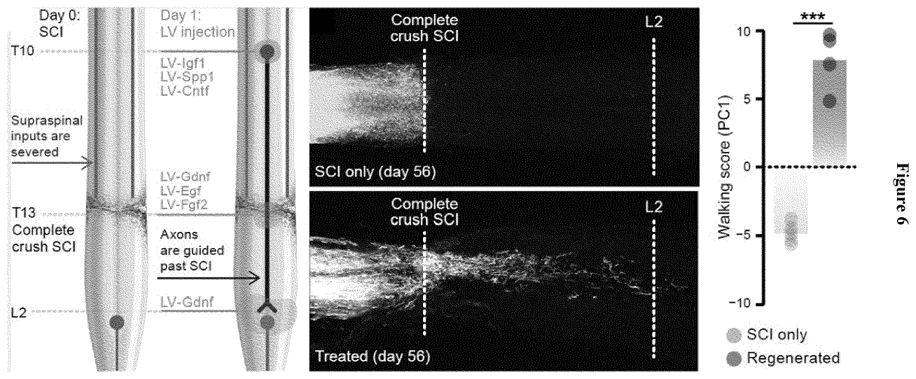

To evaluate the causal involvement of thoracic Vsx2^{ON} neurons in the recovery of walking, these neurons were ablated in cohort three by expressing the diphtheria toxin receptor (DTR) with injections of AAV5-hSyn-flex-DTR into the thoracic spinal cord of Vsx2^{Cre} mice (FIG. 5). Eight weeks after SCI plus repair strategy, all 4 mice in cohort three had regained the ability to walk with gait patterns that resembled those quantified in mice that had undergone natural repair (FIG. 5). Administration of diphtheria toxin re-paralyzed every tested mouse (FIG. 5). Post-mortem anatomical analyses confirmed the near complete ablation of Vsx2^{ON} neurons in the thoracic spinal cord (data not shown).

Together these results showed that SC^{Vsx2::Zfhx3→lumbar} neurons with regenerated axons that crossed anatomically complete SCI and reached their lumbar targets were both necessary and sufficient for the recovery of voluntary walking after complete paralysis.

In an attempt to demonstrate the usefulness single agents according to the invention in the functional recovery of incomplete SCI, the inventors performed two additional exemplary experiments described herein below.

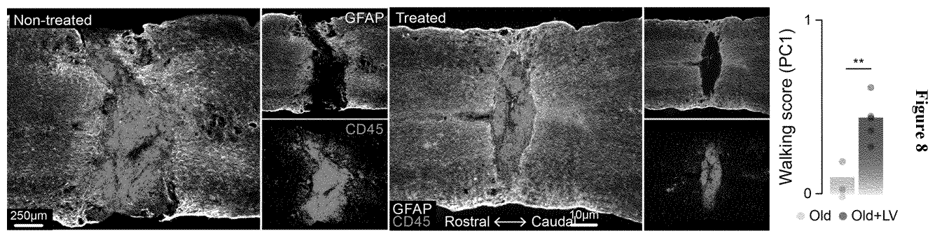

Use of the first agent of the invention alone: data shown in FIG. 7 demonstrates that neurological recovery and incomplete SCI can be improved with growth program reactivation alone (the first agent (Igf1/Spp1/Cntf)). With only the first agent, the inventors observed significantly more axon sprouting past the incomplete injury, which doubled the recovery of function (BMS Score, observational scoring system).

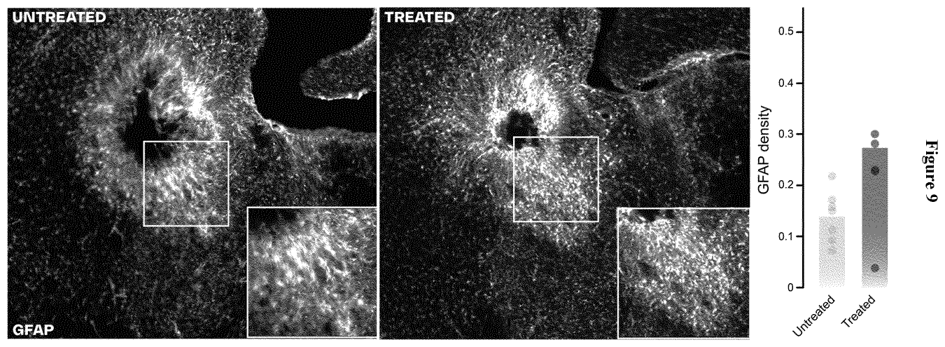

Use of the second agent of the invention alone: In the context of incomplete SCI, the inventors also demonstrated that acute intervention with viruses expressing Egf, Fgf2, Gdnf, and Vegf (the second agent) has the capacity to accelerate blood-brain-barrier reformation, astrocyte barrier formation, prevent immune infiltration, and improve neurological recovery (FIG. 8, FIG. 9).

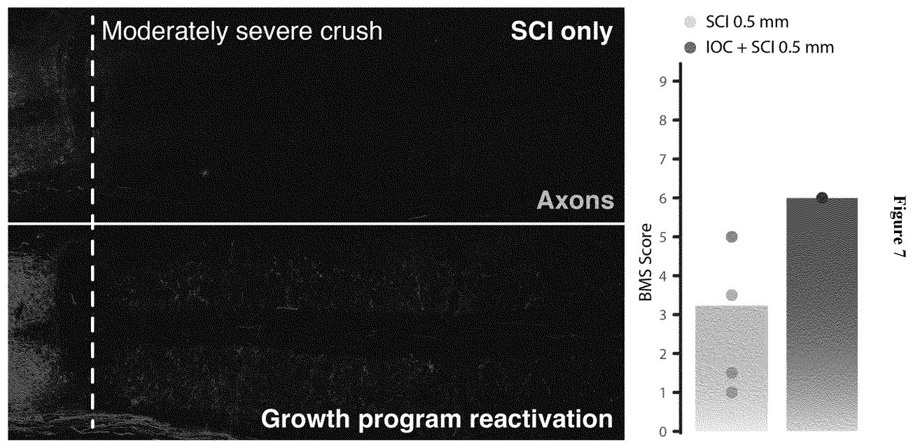

Restoration of walking using singular LV constructs: In the context of anatomically complete SCI, the inventors also demonstrated that functional recovery of walking in tested mice could be achieved through injection of Lentivirus vectors (LV) at three different locations. Rostral to the lesion, the inventors injected a cocktail of three LVs (first agent), each expressing one key growth factor that upregulates dormant developmental growth programs (respectively Igf1, Cntf, Spp1). In the lesion, the inventors injected a second agent comprising three LVs, wherein two LVs expressing different growth factors that manipulate the lesion microenvironment (respectively Fgf2 and Egf) and a third LV expressing a factor that activates chemoattraction (Gdnf). Caudal to the lesion, the inventors injected a third agent comprising only the LV expressing Gdnf. The results indicate that replacing AAVs with combinations of individual growth factor expressing LVs enables the delivery of this therapy after an SCI. It was found that this manipulation, when delivered in a single surgery, led to robust regeneration of axons through an anatomically complete SCI (see FIG. 6). The consequence of this regeneration was the gradual recovery of walking in every tested mouse.

### Methods

Mouse model. Adult male or female C57BL/6 mice (15-25 g body weight, 8-15 weeks of age) or transgenic mice were used for all experiments. Vsx2^{Cre} (MMMRRC 36672, also called Chx10^{Cre}) transgenic mouse strain was bred and maintained on a mixed genetic background (C57BL/6). Housing, surgery, behavioral experiments and euthanasia were all performed in compliance with the Swiss Veterinary Law guidelines. Manual bladder voiding and all other animal care was performed twice daily throughout the entire experiment. All procedures and surgeries were approved by the Veterinary Office of the Canton of Geneva (Switzerland; authorizations GE/25/17 and GE/109/20).

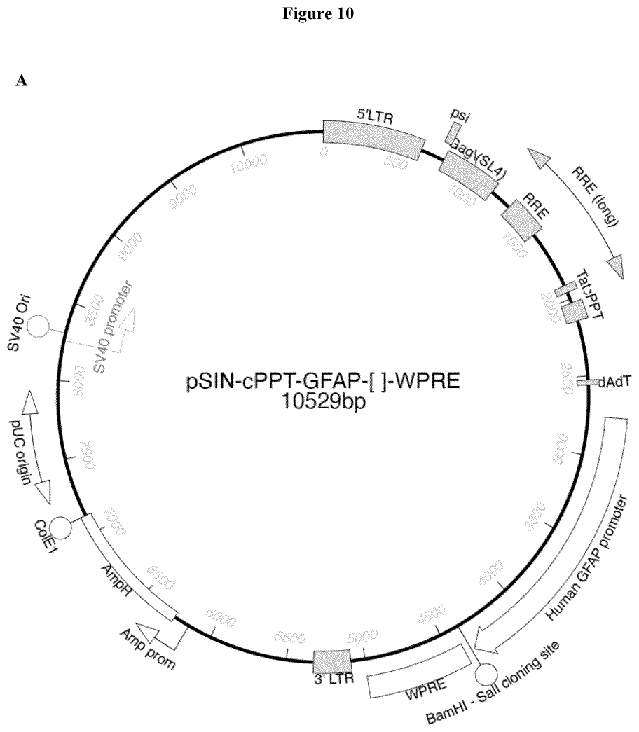

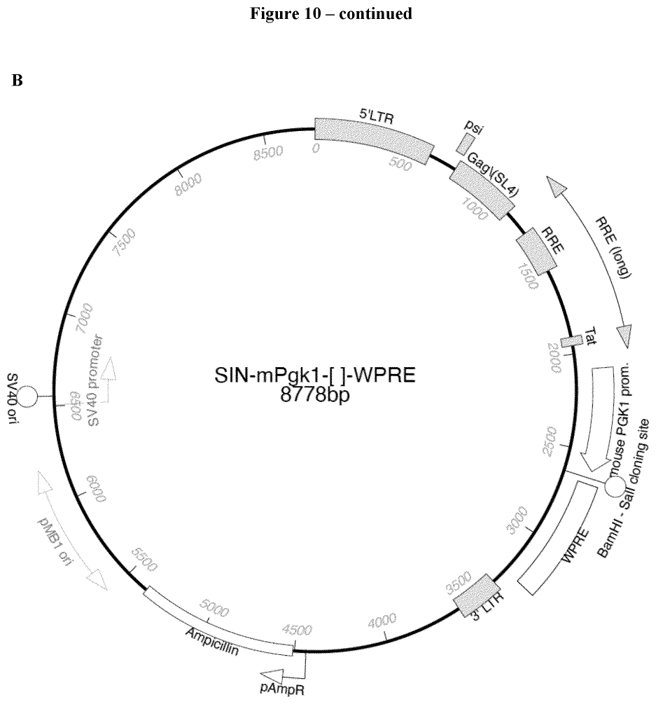

Viral vectors and vector production. Viruses used in this study were either acquired commercially or produced at the EPFL core facility. The following AAV plasmids were used: AAV5-CMV-TurboRFP (Addgene #105548), AAV9-CAG-IGF1, AAV9-CAG-Spp1, AAV9-CAG-CNTF (Igf1, Spp1, and Cntf), AAV5-CAG-COMET-GFP, AAV-CAG-flex-tdTomato, AAV-CAG-flex-human Diphtheria Toxin Receptor (DTR, rAAV2-hSyn-KASH-GFP (Addgene #60231), AAV5-hSyn-Con/Fon-eYFP (Addgene #55650), rAAV2-EF1a-DIO-Flpo (Addgene #87306) and SIN-cPPT-GFAP-GDNF-WPRE. See also FIG. 10. Injection volumes, coordinates and experimental purpose are described below.

SCI models. Spinal cord crushes were described previously. Staggered hemisection SCIs were performed as previously described. For staggered hemisection SCI, a laminectomy was made at the mid-thoracic level (T12) and the lateral half of the spinal cord was cut using a microscapel. 8 weeks after the first hemisection, a second mid-thoracic hemisection (T7) was performed on the opposite side of the first hemisection.

Biological repair intervention. General surgical procedures have been described previously in detail. Surgeries were performed under aseptic conditions and under 1-2% isoflurane in 0.5-1 L/min flow of oxygen as general anaesthesia. Surgeries were performed at EPFL under general anaesthesia with isoflurane in oxygen-enriched air using an operating microscope (Zeiss), and rodent stereotaxic apparatus (David Kopf) as previously described. AAV injections were made two weeks before SCI to allow time for molecular expression and were targeted at propriospinal neurons one and two segments rostral to the planned locations of SCI lesions after laminectomy of a single vertebra. AAVs were injected into two sites (one on each side of the cord, 0.25 μl [AAV2/9 Spp1: 1×10¹³, Igf1: 5×10¹², Cntf: 5×10¹² genome copies per ml in sterile saline]) 0.6 mm below the surface at 0.1 μl per minute using glass micropipettes connected via high-pressure tubing (Kopf) to 10-μl syringes under the control of a microinfusion pump. Severe crush SCIs were made at the level of T12/T13 after laminectomy of a single vertebra by using No. 5 Dumont forceps (Fine Science Tools) without spacers and with a tip width of 0.5 mm to completely compress the entire spinal cord laterally from both sides for 5 s. Hydrogel depots were injected stereotaxically into the center of SCI lesions 0.6 mm below the surface at 0.15 μl per minute using glass micropipettes connected via high-pressure tubing (Kopf) to 10-μl syringes under the control of microinfusion pumps, 2 days after SCI. In animals receiving 2 hydrogel depots, the second depot was placed 1.5 mm caudal to the SCI 9 days after SCI. For animals receiving 3 hydrogel depots, the third depot was made 2.5 mm below the SCI 16 days after SCI. For animals receiving injections of the lentiviral vector encoding human GDNF (LV-GDNF: 600 μg P24/mL), the vector injection was made at the L2 and L4 spinal segments 2 days after SCI. Tract-tracing was performed by injection of AAV2/5 RFP red fluorescent protein (RFP, University of Pennsylvania Vector Core, 2.612×10¹³ genome copies per ml) injected 4×0.25 μl into the segments rostral to SCI 9 days after SCI. After surgeries, mice were allowed to wake up in an incubator. Analgesia, Buprenorphine (Essex Chemie AG, Switzerland, 0.01-0.05 mg/kg s.c.) or Rimadyl (5 mg/kg s.c.), was given twice daily for 2-3 days after surgery. Animals were randomly assigned numbers and thereafter were evaluated blind to experimental conditions. Seven days after SCI, all mice were evaluated in open field and all animals exhibiting any hindlimb movements were not studied further.

Hydrogel depots with growth factors. Biomaterial depots were prepared using well-characterized diblock copolypeptide hydrogels and loaded with growth factors as previously described. Human recombinant FGF2, EGF, and GDNF were purchased from Peprotech: (i) human FGF2 (FGF-basic) (154 amino acids) Cat #100-18B-100UG, Lot #091608 C0617; (ii) human EGF Cat #AF-100-15-100UG, Lot #0816AFC05 B2317; (iii) human GDNF Cat #405-10-100UG, Lot #0606B64 A2517. Freeze-dried K₁₈₀L₂₀ powder was reconstituted to 3.0% w/v in sterile PBS with combinations of FGF2 (1.0 μg/μl), EGF (1.0 μg/μl), and GDNF (1.0 μg/μl).

Spinal injections for retrograde labeling. To retrogradely label neurons for fluorescent-activated nuclear sorting and subsequent snRNA-seq, a partial laminectomy over the L2 spinal level was performed and two sets of bilateral injections of rAAV2-hSyn-KASH-GFP were made (0.15 μl per injection) at two depths (0.8 mm and 0.4 mm below the dorsal surface) and separated by 1 mm. To label regenerating neurons, animals received viral injections four weeks after SCI and following repair intervention. Animals were perfused three weeks following viral injections. To retrogradely label regenerating neurons for histological assessment, one set of bilateral injections of CTB 647 (Thermofisher C34778) were made (0.15 μl per injection) at two depths (0.8 mm and 0.4 mm below the dorsal surface). Animals were perfused three days following CTB injections.

Spinal injections for exclusive labeling of long distance Vsx2^{ON} neurons. To exclusively label Vsx2^{ON} neurons in the mid-thoracic spinal cord with long-distance projections to the lumbar executive centers, Boolean logic viral strategies were leveraged. Partial laminectomies were made over the T10 and L2 spinal segments of Vsx2^{Cre} mice. Two sets of bilateral injections of AAV5-hSyn-Con/Fon-eYFP (Addgene #55650) were made over the T10 spinal segment (0.25 μl per injection) at a depth of 0.6 mm below the dorsal surface and separated by 1 mm. Two sets of bilateral injections of rAAV2-EF1a-DIO-F1po (Addgene #87306) were made (0.15 μl per injection) at two depths (0.8 mm and 0.4 mm below the dorsal surface) and separated by 1 mm. Animals were perfused three weeks later.

Neuron subpopulation-specific ablation. For ablation experiments with diphtheria toxin, Vsx2^{Cre} mice were subjected to biological repair as described above. Two sets of bilateral injections of AAV5-CAG-FLEX-DTR (were made over the T10 spinal segment (0.25 μl per injection) at a depth of 0.6 mm below the dorsal surface and separated by 1 mm. Two weeks after spinal infusions, mice received intraperitoneal injections of diphtheria toxin (Sigma, D0564) diluted in saline (100 μg/kg) to ablate Vsx2 neurons. Mice were tested just before ablation and one week post-ablation.

Brainstem injections. An incision was made across the skull. To target descending neurons in the ventral gigantocellular nucleus (vGi), bregma was identified and a craniotomy 5 mm-6 mm dorsal and 0 mm-2 mm lateral to Bregma was performed. 100 nL injections of AAV5-CAG-greenComet were made at 0.15 μl per minute were made bilaterally at medial-lateral 0.3 mm, rostro-caudally at −5.8 mm and −6.2 mm, dorso-ventrally at a depth of 5.6 mm from the brain surface. Animals were perfused three weeks later.

Behavioural assessments. All the behavioral procedures have been described in detail previously. During overground walking, bilateral leg kinematics were captured with twelve infrared cameras of a Vicon Motion Systems (Oxford, UK) that tracked reflective markers attached to the crest, hip, knee, ankle joints, and distal toes. The limbs were modelled as an interconnected chain of segments and a total of 80 gait parameters were calculated from the recordings. To evaluate differences between experimental conditions, as well as to identify the most relevant parameters to account for these differences, a multistep multifactorial analysis was implemented based on principal component analysis, which was described in detail previously. To assess the similarity of mice that regained walking after treatment with other experimental groups, including those that had undergone natural repair, linear discriminant analysis classification was implemented. Classifiers were trained on the kinematic parameters for each step of each mouse, for each experimental group, with the regeneration-treated mice held out. This process was completed 50 times with repeated subsamples of kinematic features. The trained classifiers were then leveraged to predict the experimental label, using the held out kinematic parameters from the mice that received the regeneration treatment. This process was repeated for the loss-of-function experiment in Vsx2^{Cre} mice.

Perfusions. Mice were perfused at the end of the experiments. Mice were deeply anesthetized by an intraperitoneal injection of 0.2 mL sodium pentobarbital (50 mg/mL). Mice were transcardially perfused with PBS followed by 4% paraformaldehyde in PBS. Tissue was removed and post-fixed overnight in 4% paraformaldehyde before being transferred to PBS or cryoprotected in 30% sucrose in PBS.

Immunohistochemistry. Immunohistochemistry was performed as described previously. Perfused post-mortem tissue was cryoprotected in 30% sucrose in PBS for 48 hours before being embedded in cryomatrix (Tissue Tek O.C.T, Sakura Finetek Europe B.V.) and freezing. 30 μm thick transverse or horizontal sections of the spinal cord were cut on a cryostat (Leica), immediately mounted on glass slides and dried or in free floating wells containing PBS+0.03% sodium azide. Primary antibodies were: rabbit anti-GFAP (1:1000; Dako); guinea pig anti NeuN (1:300; Millipore); rabbit anti-GDNF-α (GDNF-receptor alpha) (1:1000 Abcam); guinea pig anti-homer1 (1:600, Synaptic Systems Gmbh); rabbit anti-synaptophysin (1:600, Dako); chicken anti-RFP (1:500, Novus Biologicals); goat anti-GFP (1:1000, Novus Biologicals); chicken anti-GFP (1:500 Life Technologies); rabbit anti-Chx10 (also known as Vsx2) (1:500, Novus Biologicals); sheep anti-Zfhx3 (1:500, Novus Biologicals). Fluorescent secondary antibodies were conjugated to Alexa 488 (green), or Alexa 405 (blue), or Alexa 555 (red), or Alexa 647 (far red) (ThermoFisher Scientific, USA). Nuclear stain: 4′,6′-diamidino-2-phenylindole dihydrochloride (DAPI; 2 ng/ml; Molecular Probes). Sections were imaged digitally using a slide scanner (Olympus VS-120 Slide scanner) or confocal microscope (Zeiss LSM880+Airy fast module with ZEN 2 Black software (Zeiss, Oberkochen, Germany). Images were digitally processed using ImageJ (ImageJ NIH) software or Imaris (Bitplane, v.9.0.0).

Tissue clearing (CLARITY). Samples were incubated in X-CLARITY hydrogel solution (Logos Biosystems Inc., South Korea) for 24 hours at 4 degrees Celsius with gentle shaking. Samples were degassed and polymerized using the X-CLARITY Polymerisation System (Logos Biosystems Inc., South Korea), followed by washes in 0.001M PBS for 5 minutes at room temperature. Samples were next placed in the X-CLARITY Tissue Clearing System (Logos Biosystems Inc., South Korea), set to 1.5 A, 100 RPM, 37 degrees, for 29 h. Clearing solution was made in-house with 4% sodium dodecyl sulfate (SDS), 200 mM boric acid with dH₂O, pH adjusted to 8.5. Following this, samples were washed for at least 24 h at room temperature with gentle shaking in 0.1 M PBS solution containing 0.1% Triton X-100 to remove excess SDS. Finally, samples were incubated in 40 g of Histodenz dissolved in 30 mL of 0.02M PB, pH 7.5, 0.01% sodium azide (refractive index 1.465) for at least 24 h at room temperature with gentle shaking prior to imaging.

3D imaging. Imaging of cleared tissue was performed using either a customized mesoSPIM and CLARITY-optimized light-sheet microscope (COLM). A custom-built sample holder was used to secure the central nervous system in a chamber filled with RIMS. Samples were imaged using either a 1.25× or 2.5× objective at the mesoSPIM and a 4× or 10× objective at the COLM with one or two light sheets illuminating the sample from both the left and right sides. The voxel resolution in the x-, y- and z directions was 5.3 μm×5.3 μm×5 μm for the 1.25× acquisition and 2.6 μm×2.6 μm×3 μm for the 2.5× acquisition. The voxel resolution of the COLM was 1.4 μm×1.4 μm by 5 μm for the 4× and 0.59 μm×0.59 μm×3 μm for the 10× acquisition. Images were generated as 16-bit TIFF files and then stitched using Arivis Vision4D (Arivis AG, Munich, Germany). 3D reconstructions and optical sections of raw images were generated using Imaris (bitplane, V.9.8) software.

Axon quantification. To align all sections to a common coordinate space, a custom image analysis pipeline was implemented that includes preprocessing, registration and combination of histological images from different sections. In brief, all preprocessing was implemented in Fiji, and all registration procedures in R, using the image analysis package ‘imageR’, and medical image registration package ‘RNiftyReg’. Images were aligned to a template spinal cord section. Axon densities were calculated for 200 μm bins, beginning at the lesion epicentre.

Cell counts. To quantify the proportion of neurons expressing Vsx2 and/or Zfhx3, the number of NeuN positive neurons expressing Vsx2 and/or Zfhx3 was counted. Cell counts were performed using the image analysis software Imaris (bitplane, V.9.8).

Synapses detection. To detect the number of synapses contacting neurons, the surface of the cells was first reconstructed using the surface reconstruction module in Imaris (bitplane, V9.8) at a 10 μm resolution. The synapses were then identified using spot detection in Imaris. To identify the synapses in close apposition to the neurons of interest, a MATLAB algorithm was used to segregate the synapses located with a distance between 0 and 1 um to the reconstructed surface of the neurons.

Electrophysiology. Animals were anesthetized with ketamine/xylazine, a small burr hole was drilled in the skull to provide access to the vGi and needle electrodes were inserted into the tibialis anterior muscle of both the left and and right hindlimb. A platinum/iridium concentric bipolar electrode (PI-SNE-100, Microprobes USA) was inserted into the vGi (coordinates: −6.0 AP, 0.3 ML, −5.7 DV relative to bregma). Electrical stimulation (STG4000, Multi Channel Systems) was delivered to the vGi in trains of 5×200 us square wave pulses at 500 Hz, repeated once every 5 seconds. Stimulation was delivered at 20, 40, 60, 80 and 100 μA, with 10 repetitions of each intensity. Evoked EMG recordings were amplified (1000×) and filtered (300 Hz high pass, 5 kHz low pass) using a differential amplifier (model 1700, AM Systems) before being digitized and recorded (PowerLab 8/35, AD Instruments). Mean peak to peak amplitudes of evoked responses were calculated offline (LabChart Pro, AD instruments) and compared between groups.

Statistics, power calculations, group sizes and reproducibility. Statistical evaluations of repeated measures were conducted by one-way ANOVA with post hoc independent pairwise analysis as per TukeyHSD. Power calculations were performed using G*Power Software v.3.1.9.245. For quantification of histologically derived neuroanatomical outcomes such as axons density, group sizes were used that were calculated to provide at least 80% power when using the following parameters: probability of type I error (a)=0.05, a conservative effect size of 0.25, 3-10 treatment groups with multiple measurements obtained per replicate. All graphs show mean±s.e.m. as well as individual values as dot plots. All bar graphs are overlaid with dot plots in which each dot represents the value for one animal. Experiments testing axon regrowth across SCI lesions in animals were repeated independently at least twice in different groups of mice with similar results. For all photomicrographs of histological tissue, staining experiments were repeated independently with tissue from at least four, and in most cases six, different animals with similar results.

Single-nucleus RNA sequencing. Single-nucleus dissociation of the mouse lumbar spinal cord was performed according to our established procedures. Following euthanasia by isoflurane inhalation and cervical dislocation, the lumbar spinal cord site was immediately dissected and frozen on dry ice. Spinal cords were doused in 500 μl sucrose buffer (0.32 M sucrose, 10 mM HEPES [pH 8.0], 5 mM CaCl₂), 3 mM Mg acetate, 0.1 mM EDTA, 1 mM DTT) and 0.1% Triton X-100 with the Kontes Dounce Tissue Grinder. 2 mL of sucrose buffer was then added and filtered through a 40-μm cell strainer. The lysate was centrifuged at 3200 g for 10 min at 4° C. The supernatant was then decanted, and 3 mL of sucrose buffer was added to the pellet for 1 min. The pellet was homogenized using an Ultra-Turrax and 12.5 mL of density buffer (1 M sucrose, 10 mM HEPES [pH 8.0], 3 mM Mg acetate, 1 mM DTT) was added below the nuclei layer. The tube was centrifuged at 3200 g at 4° C. and supernatant poured off. Nuclei on the bottom half of the tube wall were collected with 100 μl PBS with 0.04% BSA and 0.2 U/μl RNase inhibitor. Finally, nuclei were resuspended through a 30 μm strainer, and adjusted to 1000 nuclei/μl.

Projection-specific snRNA-seq. For projection-specific snRNA-seq experiments the nuclei were first resuspended in 500 μl Pre-FACS buffer (1×PBS with 1% BSA, 0.2 U/μl SUPERaseIn RNase Inhibitor), and filtered through a 35 μm cell strainer. Samples were processed on a Sony SH800 Cell Sorter with a 100 mm sorting chip. Nuclei were gated using forward scatter, side scatter and DRAQ5+ measurements to ensure that doublets were gated out. After this initial gating, GFP+ nuclei were identified using a two-dimensional scatterplot. GFP+/DRAQ5+ nuclei were collected into 1.5 ml centrifuge tubes containing 10 μl of the Pre-FACS buffer. ~500 GFP+ nuclei were collected from the uninjured spinal cord (these nuclei were pooled from n=5 mice) and ~800 GFP+ nuclei from regenerating axons (these nuclei were pooled from n=13 mice). GFP+ nuclei were then loaded directly onto a Chromium Single Cell Processor (10× Genomics) for barcoding of RNA from single nuclei.

Library preparation. snRNA-seq library preparation was carried out using the 10× Genomics Chromium Single Cell Kit Version 3. The nuclei suspension was added to the Chromium RT mix to achieve loading numbers of 2000-5000. For downstream cDNA synthesis (13 PCR cycles), library preparation and sequencing, the manufacturer's instructions were followed.

Read alignment. Reads were aligned to the most recent Ensembl release (GRCm38.93) using Cell Ranger, and obtained a matrix of unique molecular identifier (UMI) counts. Seurat was used to calculate quality control metrics for each cell barcode, including the number of genes detected, number of UMIs, and proportion of reads aligned to mitochondrial genes. Low-quality cells were filtered by removing cells expressing less than 200 genes or with more than 5% mitochondrial reads. Genes expressed in less than three cells were likewise removed.

Clustering and integration. Prior to clustering analysis, batch effect correction and data integration across the two different experimental conditions as previously described were first performed. Gene expression data was normalized using regularized negative binomial models, then integrated across batches using the data integration workflow within Seurat. The normalized and integrated gene expression matrices were then subjected to clustering to identify cell types in the integrated dataset, again using the default Seurat workflow. Cell types were manually annotated on the basis of marker gene expression, guided by previous studies of the mouse spinal cord. Local and projecting neuronal subpopulations were annotated on the basis of Nfib and Zfhx3 expression, respectively. Following our projection-specific snRNA-seq experiment in uninjured mice, each subsequent experiment was re-integrated with this dataset prior to subpopulation annotation. This enabled the identification of the same 28 neuronal subpopulations across the three distinct experiments.

Cell type prioritization with Augur. To identify neuronal subpopulations perturbed during natural repair, inventors' machine-learning method Augur was implemented. Augur was run with default parameters for all comparisons. To evaluate the robustness of cell type prioritizations to the resolution at which neuronal subtypes were defined in the snRNA-seq data, Augur was applied at various clustering resolutions, and visualized the resulting cell type prioritizations both on a hierarchical clustering tree of neuron subtypes and as a progression of UMAPs. The key assumption underlying Augur is that cell types undergoing a profound response to a perturbation should become more separable, within the highly multidimensional space of gene expression, than less affected cell types. Briefly, Augur withholds a proportion of sample labels, then trains a random forest classifier to predict the condition from which each cell was obtained. The accuracy with which this prediction can be made from single-cell gene expression measurements is then evaluated in cross-validation, and quantified using the area under the receiver operating characteristic curve (AUC).

Cell type proportions. To compare the proportion of neuronal subpopulations within and between datasets, the normalized proportion of each neuronal subpopulation was calculated. The distributions were compared to the expected proportion in the uninjured dataset using Chi-squared test.

1. A pharmaceutical combination comprising:
i) a first agent comprising exogenous nucleic acid encoding an osteopontin (Spp1) polypeptide as set forth in SEQ ID NO: 1 or SEQ ID NO: 8, insulin-like growth factor 1 (Igf1) polypeptide as set forth in SEQ ID NO: 2, and ciliary-derived neurotrophic factor (Cntf) polypeptide as set forth in SEQ ID NO: 3 or SEQ ID NO: 9; and/or
ii) a second agent comprising exogenous nucleic acid encoding a fibroblast growth factor 2 (FGF2) polypeptide as set forth in SEQ ID NO: 4 or SEQ ID NO: 10, epidermal growth factor (EGF) polypeptide as set forth in SEQ ID NO: 5 or SEQ ID NO: 11, and a glial-derived neurotrophic factor (GDNF) polypeptide as set forth in SEQ ID NO: 6;
wherein, when both present, said first and second agents are administered sequentially or simultaneously, and
wherein said first and second agents provide sustained expression and secretion of effective amounts of said polypeptides during at least 3 days.

2. The pharmaceutical combination of claim 1, wherein the second agent further comprises exogenous nucleic acid encoding a vascular endothelial growth factor (VEGF) polypeptide as set forth in SEQ ID NO: 7 or SEQ ID NO: 12.

3. The pharmaceutical combination of claim 1, further comprising:
iii) a third agent comprising exogenous nucleic acid encoding a glial-derived neurotrophic factor (GDNF) polypeptide as set forth in SEQ ID NO: 6;
wherein said first, second and third agents are administered sequentially or simultaneously, and
wherein said first, second and third agents provide sustained expression and secretion of effective amounts of said polypeptides during at least 3 days.

4. The pharmaceutical combination of claim 1, wherein the first agent, the second agent, and the third agent are a gene editing systems, a vector or expression vectors containing one or more nucleic acid sequences encoding the one or more polypeptides according to claim 1.

5. The pharmaceutical combination of claim 4, wherein the expression vector is a viral vector selected from the group consisting of retrovirus, lentivirus, adenovirus, herpesvirus, poxvirus, alpha virus, vaccinia virus, and an adeno-associated viruses.

6. A method of treating an incomplete spinal cord injury or a stroke in a subject in need thereof, the method comprising administering the pharmaceutical combination of claim 1 to the subject.

7. A method of treating a complete spinal cord injury in a subject in need thereof, the method comprising administering the pharmaceutical combination of claim 3 to the subject, wherein the pharmaceutical combination comprises the first agent, the second agent and the third agent.

8. The method of claim 6, wherein the incomplete spinal cord injury is an injury of the cervical, thoracic, and/or lumbar spinal cord.

9. The method of claim 6, wherein the treatment of stroke is neuronal, functional, and anatomical repair of brain after a stroke.

10. The method of claim 8, wherein repair of the incomplete spinal cord injury or stroke is obtained by targeting V1 neurons, V0 neurons, V2b neurons, CSF contacting neurons, Vglut2 neurons, Vgat neurons, Chat neurons, Vsx2, and/or Zfhx3 (projection V2a neurons).

11. The method of claim 8, wherein repair of the incomplete spinal cord injury consists of axon regeneration and restoring neurological functions.

12. The method of claim 6, wherein the method of treating further comprises assessing whether the subject suffers from an incomplete spinal cord injury, a complete spinal cord injury, or a stroke.

13. The method of claim 12, wherein the assessing comprises analysis of spinal cord injury symptoms or stroke symptoms comprising one or more of loss or impairment of motor function(s), loss or impairment of sensory function(s), and loss or impairment of autonomic function(s), in the cervical, thoracic, lumbar, or sacral segments of the spinal cord, and/or the brain.

14. The method of claim 7, wherein the first agent, the second agent and the third agent of the pharmaceutical combination or the pharmaceutical combination are administered by injection into the cervical, thoracic, lumbar, or sacral spinal cord of a subject, or brain, via stereotaxic injection, or by intravenous infusion or intravenous injection.

15. The method of claim 14, wherein the stereotaxic injection is guided by localization of the spinal cord injury epicenter with a methods comprising ultrasound guidance, electrophysiology, or molecular guidance.

16. The method of claim 6, wherein the subject is a mammal.

17. The method of claim 7, wherein the complete spinal cord injury is an injury of the cervical, thoracic, and/or lumbar spinal cord.

18. The method of claim 17, wherein repair of the complete spinal cord injury is obtained by targeting V1 neurons, V0 neurons, V2b neurons, CSF contacting neurons, Vglut2 neurons, Vgat neurons, Chat neurons, Vsx2, and/or Zfhx3 (projection V2a neurons).

19. The method of claim 7, wherein repair of the complete spinal cord injury consists of axon regeneration and restoring neurological functions.

20. The method of claim 7, wherein the method of treating further comprises assessing whether the subject suffers from an incomplete spinal cord injury, a complete spinal cord injury, or a stroke.

21. The method of claim 20, wherein the assessing comprises analysis of spinal cord injury symptoms or stroke symptoms comprising one or more of loss or impairment of motor function(s), loss or impairment of sensory function(s), and loss or impairment of autonomic function(s), in the cervical, thoracic, lumbar, or sacral segments of the spinal cord and/or the brain.

22. The method of claim 7, wherein the subject is a mammal.


SEQUENCE LISTING (12 sequences)

SEQ ID NO: 1 - protein, 302 aa, Mus musculus
MRLAVICFCLFGIASSLPVKVTDSGSSEEKLYSLHPDPIATWLVPDPSQKQNLLAPQNAVSSEEKDDFKQETLPSNSNESHDHMDDDDDDDDDDGDHAESEDSVDSDESDESHHSDESDETVTASTQADTFTPIVPTVDVPNGRGDSLAYGLRSKSRSFQVSDEQYPDATDEDLTSHMKSGESKESLDVIPVAQLLSMPSDQDNNGKGSHESSQLDEPSLETHRLEHSKESQESADQSDVIDSQASSKASLEHQSHKFHSHKDKLVLDPKSKEDDRYLKFRISHELESSSSEVNDYKDDDDK

SEQ ID NO: 2 - protein, 153 aa, Homo sapiens
MGKISSLPTQLFKCCFCDFLKVKMHTMSSSHLFYLALCLLTFTSSATAGPETLCGAELVDALQFVCGDRGFYFNKPTGYGSSSRRAPQTGIVDECCFRSCDLRRLEMYCAPLKPAKSARSVRAQRHTDMPKTQKEVHLKNASRGSAGNKNYRM

SEQ ID NO: 3 - protein, 207 aa, Mus musculus
MAFAEQSPLTLHRRDLCSRSIWLARKIRSDLTALMESYVKHQGLNKNISLDSVDGVPVASTDRWSEMTEAERLQENLQAYRTFQGMLTKLLEDQRVHFTPTEGDFHQAIHTLTLQVSAFAYQLEELMALLEQKVPEKEADGMPVTIGDGGLFEKKLWGLKVLQELSQWTVRSIHDLRVISSHHMGISAHESHYGAKQMYPYDVPDYA

SEQ ID NO: 4 - protein, 154 aa, Mus musculus
MAASGITSLPALPEDGGAAFPPGHFKDPKRLYCKNGGFFLRIHPDGRVDGVREKSDPHVKLQLQAEERGVVSIKGVCANRYLAMKEDGRLLASKCVTEECFFFERLESNNYNTYRSRKYSSWYVALKRTGQYKLGSKTGPGQKAILFLPMSAKS

SEQ ID NO: 5 - protein, 1228 aa, Mus musculus
MAPKKKRKVRIMPWGRRPTWLLLAFLLVFLKISILSVTAWQTGNCQPGPLERSERSGTCAGPAPFLVFSQGKSISRIDPDGTNHQQLVVDAGISADMDIHYKKERLYWVDVERQVLLRVFLNGTGLEKVCNVERKVSGLAIDWIDDEVLWVDQQNGVITVTDMTGKNSRVLLSSLKHPSNIAVDPIERLMFWSSEVTGSLHRAHLKGVDVKTLLETGGISVLTLDVLDKRLFWVQDSGEGSHAYIHSCDYEGGSVRLIRHQARHSLSSMAFFGDRIFYSVLKSKAIWIANKHTGKDTVRINLHPSFVTPGKLMVVHPRAQPRTEDAAKDPDPELLKQRGRPCRFGLCERDPKSHSSACAEGYTLSRDRKYCEDVNECATQNHGCTLGCENTPGSYHCTCPTGFVLLPDGKQCHELVSCPGNVSKCSHGCVLTSDGPRCICPAGSVLGRDGKTCTGCSSPDNGGCSQICLPLRPGSWECDCFPGYDLQSDRKSCAASGPQPLLLFANSQDIRHMHFDGTDYKVLLSRQMGMVFALDYDPVESKIYFAQTALKWIERANMDGSQRERLITEGVDTLEGLALDWIGRRIYWTDSGKSVVGGSDLSGKHHRIIIQERISRPRGIAVHPRARRLFWTDVGMSPRIESASLQGSDRVLIASSNLLEPSGITIDYLTDTLYWCDTKRSVIEMANLDGSKRRRLIQNDVGHPFSLAVFEDHLWVSDWAIPSVIRVNKRTGQNRVRLQGSMLKPSSLVVVHPLAKPGADPCLYRNGGCEHICQESLGTARCLCREGFVKAWDGKMCLPQDYPILSGENADLSKEVTSLSNSTQAEVPDDDGTESSTLVAEIMVSGMNYEDDCGPGGCGSHARCVSDGETAECQCLKGFARDGNLCSDIDECVLARSDCPSTSSRCINTEGGYVCRCSEGYEGDGISCFDIDECQRGAHNCAENAACTNTEGGYNCTCAGRPSSPGRSCPDSTAPSLLGEDGHHLDRNSYPGCPSSYDGYCLNGGVCMHIESLDSYTCNCVIGYSGDRCQTLDLRWWELRHAGYGQKHDIMVVAVCMVALVLLLLLGMWGTYYYRTRKQLSNPPKNPCDEPSGSVSSSGPDSSSGAAVASCPQPWFVVLEKHQDPKNGSLPADGTNGAVVDAGLSPSLQLGSVHLTSWRQKPHIDGMGTGQSCWIPPSSDRGPQEIEGNSHLPSYRPVGPEKLHSLQSANGSCHERAPDLPRQTEPVQ

SEQ ID NO: 6 - protein, 211 aa, Homo sapiens
MKLWDVVAVCLVLLHTASAFPLPAGKRPPEAPAEDRSLGRRRAPFALSSDSNMPEDYPDQFDDVMDFIQATIKRLKRSPDKQMAVLPRRERNRQAAAANPENSRGKGRRGQRGKNRGCVLTAIHLNVTDLGLGYETKEELIFRYCSGSCDAAETTYDKILKNLSRNRRLVSDKVGQACCRPIAFDDDLSFLDDNLVYHILRKHSAKRCGCI

SEQ ID NO: 7 - protein, 214 aa, Mus musculus
MNFLLSWVHWTLALLLYLHHAKWSQAAPTTEGEQKSHEVIKFMDVYQRSYCRPIETLVDIFQEYPDEIEYIFKPSCVPLMRCAGCCNDEALECVPTSESNITMQIMRIKPHQSQHIGEMSFLQHSRCECRPKKDRTKPEKKSVRGKGKGQKRKRKKSRFKSWSVHCEPCSERRKHLFVQDPQTCKCSCKNTDSRCKARQLELNERTCRCDKPRR

SEQ ID NO: 8 - protein, 314 aa, Homo sapiens
MRIAVICFCLLGITCAIPVKQADSGSSEEKQLYNKYPDAVATWLNPDPSQKQNLLAPQNAVSSEETNDFKQETLPSKSNESHDHMDDMDDEDDDDHVDSQDSIDSNDSDDVDDTDDSHQSDESHHSDESDELVTDFPTDLPATEVFTPVVPTVDTYDGRGDSVVYGLRSKSKKFRRPDIQYPDATDEDITSHMESEELNGAYKAIPVAQDLNAPSDWDSRGKDSYETSQLDDQSAETHSHKQSRLYKRKANDESNEHSDVIDSQELSKVSREFHSHEFHSHEDMLVVDPKSKEEDKHLKFRISHELDSASSEVN

SEQ ID NO: 9 - protein, 200 aa, Homo sapiens
MAFTEHSPLTPHRRDLCSRSIWLARKIRSDLTALTESYVKHQGLNKNINLDSADGMPVASTDQWSELTEAERLQENLQAYRTFHVLLARLLEDQQVHFTPTEGDFHQAIHTLLLQVAAFAYQIEELMILLEYKIPRNEADGMPINVGDGGLFEKKLWGLKVLQELSQWTVRSIHDLRFISSHQTGIPARGSHYIANNKKM

SEQ ID NO: 10 - protein, 155 aa, Homo sapiens [sequence omitted]

SEQ ID NO: 11 - protein, 1207 aa, Homo sapiens
MLLTLIILLPVVSKFSFVSLSAPQHWSCPEGTLAGNGNSTCVGPAPFLIFSHGNSIFRIDTEGTNYEQLVVDAGVSVIMDFHYNEKRIYWVDLERQLLQRVFLNGSRQERVCNIEKNVSGMAINWINEEVIWSNQQEGIITVTDMKGNNSHILLSALKYPANVAVDPVERFIFWSSEVAGSLYRADLDGVGVKALLETSEKITAVSLDVLDKRLFWIQYNREGSNSLICSCDYDGGSVHISKHPTQHNLFAMSLFGDRIFYSTWKMKTIWIANKHTGKDMVRINLHSSFVPLGELKVVHPLAQPKAEDDTWEPEQKLCKLRKGNCSSTVCGQDLQSHLCMCAEGYALSRDRKYCEDVNECAFWNHGCTLGCKNTPGSYYCTCPVGFVLLPDGKRCHQLVSCPRNVSECSHDCVLTSEGPLCFCPEGSVLERDGKTCSGCSSPDNGGCSQLCVPLSPVSWECDCFPGYDLQLDEKSCAASGPQPFLLFANSQDIRHMHFDGTDYGTLLSQQMGMVYALDHDPVENKIYFAHTALKWIERANMDGSQRERLIEEGVDVPEGLAVDWIGRRFYWTDRGKSLIGRSDLNGKRSKIITKENISQPRGIAVHPMAKRLFWTDTGINPRIESSSLQGLGRLVIASSDLIWPSGITIDFLTDKLYWCDAKQSVIEMANLDGSKRRRLTQNDVGHPFAVAVFEDYVWFSDWAMPSVMRVNKRTGKDRVRLQGSMLKPSSLVVVHPLAKPGADPCLYQNGGCEHICKKRLGTAWCSCREGFMKASDGKTCLALDGHQLLAGGEVDLKNQVTPLDILSKTRVSEDNITESQHMLVAEIMVSDQDDCAPVGCSMYARCISEGEDATCQCLKGFAGDGKLCSDIDECEMGVPVCPPASSKCINTEGGYVCRCSEGYQGDGIHCLDIDECQLGEHSCGENASCTNTEGGYTCMCAGRLSEPGLICPDSTPPPHLREDDHHYSVRNSDSECPLSHDGYCLHDGVCMYIEALDKYACNCVVGYIGERCQYRDLKWWELRHAGHGQQQKVIVVAVCVVVLVMLLLLSLWGAHYYRTQKLLSKNPKNPYEESSRDVRSRRPADTEDGMSSCPQPWFVVIKEHQDLKNGGQPVAGEDGQAADGSMQPTSWRQEPQLCGMGTEQGCWIPVSSDKGSCPQVMERSFHMPSYGTQTLEGGVEKPHSLLSANPLWQQRALDPPHQMELTQ

SEQ ID NO: 12 - protein, 232 aa, Homo sapiens
MNFLLSWVHWSLALLLYLHHAKWSQAAPMAEGGGQNHHEVVKFMDVYQRSYCHPIETLVDIFQEYPDEIEYIFKPSCVPLMRCGGCCNDEGLECVPTEESNITMQIMRIKPHQGQHIGEMSFLQHNKCECRPKKDRARQEKKSVRGKGKGQKRKRKKSRYKSWSVYVGARCCLMPWSLPGPHPCGPCSERRKHLFVQDPQTCKCSCKNTDSRCKARQLELNERTCRCDKPRR